(19) United States
(12) Patent Application Publication
(10) Pub. No.: US 2023/0087865 A1
(43) Pub. Date: Mar. 23, 2023

(54) PHARMACEUTICAL COMPOSITION FOR ORAL ADMINISTRATION

(71) Applicant: HK INNO. N CORPORATION, Cheongju-si, Chungcheongbuk-do (KR)

(72) Inventors: Tae Keun CHO, Namdong-gu Incheon (KR); Young Dae CHO, Osan-si Gyeonggi-do (KR); Eun Kyung JEON, Hwaseong-si Gyeonggi-do (KR); Bong Tae KIM, Hanam-si Gyeonggi-do (KR); Sung Jun KIM, Hwaseong-si Gyeonggi-do (KR); Joo Hwan KIM, Seongham-si Gyeonggi-do (KR); Tae Hyeong KIM, Seoul (KR)

(21) Appl. No.: 17/802,078

(22) PCT Filed: Feb. 26, 2021
(86) PCT No.: PCT/IB2021/051606

§ 371 Date: Aug. 24, 2022

(30) Foreign Application Priority Data
Feb. 27, 2020 (KR) 10-2020-0024657
Apr. 8, 2020 (KR) 10-2020-0043047

(51) Int. Cl. A61K 31/4184; A61K 9/48; A61K 45/06; A61K 31/4365; A61K 9/20

(52) U.S. Cl. CPC A61K 31/4184; A61K 9/48; A61K 45/06; A61K 31/4365; A61K 9/20

(57) ABSTRACT

The present disclosure relates to a pharmaceutical composition that delayed-releases tegoprazan and releases clopidogrel immediately. The pharmaceutical composition may exhibit significantly excellent effects on the prevention and treatment of gastrointestinal disorders caused by administration of clopidogrel and thrombosis-related diseases.

### TECHNICAL FIELD

The present disclosure relates to a pharmaceutical composition containing clopidogrel and a compound of the following Formula 1, and more particularly, to a stable pharmaceutical composition which maintains the efficacy of clopidogrel while preventing or reducing clopidogrel-related gastrointestinal disorders by using clopidogrel in concomitant administration with a compound of the following Formula 1.

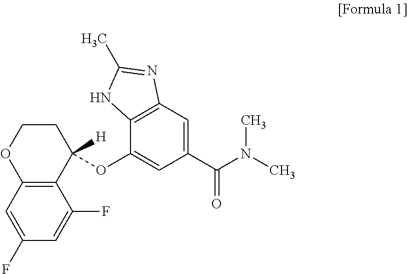 [SMILES: Cc1nc2cc(C(=O)N(C)C)cc(O[C@H]3CCOc4cc(F)cc(F)c43)c2[nH]1]

### BACKGROUND ART

Clopidogrel is a platelet aggregation inhibitor that is effective for the treatment of stroke, thrombosis, embolism, and peripheral or coronary artery diseases such as myocardial infarction, and has a chemical name of methyl (+)-(S)-α-(2-chlorophenyl)-6,7-dihydrothieno[3,2-c]pyridine-5(4H)-acetate. Clopidogrel specifically inhibits adenosine diphosphate (hereinafter referred to as “ADP”)—induced platelet aggregation by direct inhibition of ADP binding to the ADP receptor known to play an important role in thrombogenesis and direct inhibition of subsequent ADP-mediated activation of the glycoprotein GPIIb/IIa complex.

In addition, clopidogrel inhibits platelet aggregation induced by agonists other than ADP by blocking the amplification of platelet activation by released ADP. Once clopidogrel acts on platelets, it will exhibit an inhibitory effect against platelet aggregation for the lifespan of the platelets (about 7 days).

This effect of clopidogrel is an effect exerted by the active metabolite of clopidogrel.

That is, an enzyme responsible for the metabolism of clopidogrel in the liver is an important factor for the efficacy of clopidogrel. Clopidogrel was initially expected to be metabolized only by CYP1A, but the most recent study revealed that CYP2C19 is an additional enzyme that is involved in the conversion of clopidogrel into an active metabolite.

Meanwhile, clopidogrel has side effects that cause gastrointestinal disorders such as ulcers and gastrointestinal bleeding. Patients in need of long term antiplatelet drug therapy often may interrupt or not receive such therapy due to gastrointestinal disorders, and as a result, patients are deprived of beneficial therapeutic effects.

Therefore, there is a need for a method making it possible to sufficiently enjoy the beneficial therapeutic effect of clopidogrel without gastrointestinal disorders by overcoming the above-described problems of clopidogrel.

### PRIOR ART DOCUMENTS

### Patent Documents

(Patent Document 1) Korean Patent Publication No. 10-2008-0112361
(Patent Document 2) Korean Patent Publication No. 10-2015-0105419
(Patent Document 3) Korean Patent No. 10-1088247
(Patent Document 4) US Patent No. US2015/0079169

### Non-Patent Documents

(Non-Patent Document 1) Cytochrome P450 2C19 loss-of-function polymorphism is a major determinant of clopidogrel responsiveness in healthy subjects. Jean-Sebastein et al., The American Society of Hematology, Blood, 1 Oct. 2006. Vol 108, Number 7.

(Non-Patent Document 2) Drug Interaction between Proton Pump Inhibitors and Clopidogrel: Safe Perspective, Korean Journal of Internal Medicine: Vol. 81, No. 1, 2011.

### DISCLOSURE

### Technical Problem

An object of the present disclosure is to provide a pharmaceutical composition including: a first compartment including a first active ingredient; and a second compartment including a second active ingredient, wherein the first active ingredient is delayed-released, wherein the second active ingredient is released immediately; wherein the first active ingredient is tegoprazan, an optical isomer thereof, a pharmaceutically acceptable salt thereof, a hydrate or solvate thereof, or a mixture thereof; and wherein the second active ingredient is clopidogrel, an optical isomer thereof, a pharmaceutically acceptable salt thereof, a hydrate or solvate thereof, or a mixture thereof.

An object of the present disclosure is to provide a complex including: a first compartment including a first active ingredient; a second compartment including a second active ingredient; and an isolation layer preventing contact between the first active ingredient and the second active ingredient, wherein the first active ingredient is tegoprazan, an optical isomer thereof, a pharmaceutically acceptable salt thereof, a hydrate or solvate thereof, or a mixture thereof, and wherein the second active ingredient is clopidogrel, an optical isomer thereof, a pharmaceutically acceptable salt thereof, a hydrate or solvate thereof, or a mixture thereof.

An object of the present disclosure is to provide a pharmaceutical composition for preventing or treating gastrointestinal disorders caused by administration of clopidogrel, an optical isomer thereof, a pharmaceutically acceptable salt thereof, a hydrate or solvate thereof, or a mixture thereof, including tegoprazan, an optical isomer thereof, a pharmaceutically acceptable salt thereof, a hydrate or solvate thereof, or a mixture thereof.

An object of the present disclosure is to provide a pharmaceutical composition for inhibiting gastric acid secretion, including tegoprazan, an optical isomer thereof, a pharmaceutically acceptable salt thereof, a hydrate or solvate thereof, or a mixture thereof, wherein the pharmaceutical composition is co-administered with clopidogrel, an optical isomer thereof, a pharmaceutically acceptable salt thereof, a hydrate or solvate thereof, or a mixture thereof.

### Technical Solution

Hereinafter, the present disclosure will be described in detail.

Each description and embodiment disclosed in the present disclosure may be applied to each other description and embodiment. That is, all combinations of the various elements disclosed in the present disclosure fall within the scope of the present disclosure. In addition, the scope of the present disclosure is not construed as being limited by the specific description described below. In addition, it cannot be seen that the scope of the present disclosure is limited by the specific description described below.

In addition, as used herein, terms such as “first” and “second”, “upper” and “lower”, etc. are used only for distinction, and are not construed as specifying an order or a position.

The present disclosure provides a pharmaceutical composition and a protection method, which are able to protect the gastrointestinal tract from side effects associated with antiplatelet therapy by using an oral dosage form described in the present disclosure.

The present disclosure also provides a composition and a protection method, which are able to protect the gastrointestinal tract from side effects associated with antiplatelet therapy by co-administering (concomitant administration) an antiplatelet agent with an acid inhibitor.

The present disclosure provides a pharmaceutical composition including: a first compartment including a first active ingredient; and a second compartment including a second active ingredient, wherein the first active ingredient is delayed-released, and the second active ingredient is released immediately; wherein the first active ingredient is tegoprazan, an optical isomer thereof, a pharmaceutically acceptable salt thereof, a hydrate or solvate thereof, or a mixture thereof; and wherein the second active ingredient is clopidogrel, an optical isomer thereof, a pharmaceutically acceptable salt thereof, a hydrate or solvate thereof, or a mixture thereof.

The pharmaceutical composition of the present disclosure may prevent or treat gastrointestinal disorders caused by administration to clopidogrel, an optical isomer thereof, a pharmaceutically acceptable salt thereof, a hydrate or solvate thereof, or a mixture thereof, which is the second active ingredient, and does not cause a decrease in the efficacy of clopidogrel, which is a problem with conventional acid secretion inhibitors.

In addition, in the pharmaceutical composition of the present disclosure, the first active ingredient is delayed-released and the second active ingredient is released immediately. Specifically, the first active ingredient may be dissolved in intestinal fluid without being dissolved in gastric juice. Thus, when the pharmaceutical composition is administered, the second active ingredient shows a significantly higher blood concentration than the case in which both the first and second active ingredients are immediately released. As a result, the therapeutic effect of the second active ingredient is may be significantly excellent. Accordingly, the pharmaceutical composition of the present disclosure may effectively prevent or treat gastrointestinal disorders, for example, ulcers and gastrointestinal bleeding, which are the side effects of the second active ingredients, while exhibiting a better antithrombotic effect than a conventional composition in which both the first and second active ingredients are immediately released.

Therefore, since the pharmaceutical composition of the present disclosure has no interaction between tegoprazan (first active ingredient) and clopidogrel (second active ingredient), the two ingredients may exhibit sufficient pharmacological effects. Thus, clopidogrel in the pharmaceutical composition may exhibit a high bioavailability similar to when used a single dosage form, and exert a sufficient antithrombotic effect. Thus, the pharmaceutical composition of the present disclosure may effectively prevent or treat thrombosis-related diseases (stroke, thrombosis, embolism, and peripheral or coronary artery diseases such as myocardial infarction). In addition, the pharmaceutical composition may sufficiently inhibit gastric acid secretion, and thus has significantly excellent effects on the prevention or treatment of diseases mediated by acid pump antagonistic activity, such as gastrointestinal tract disease, gastroesophageal disease, gastroesophageal reflux disease (GERD), peptic ulcer, gastric ulcer, duodenal ulcer, NSAID-induced ulcer, gastritis, Helicobacter pylori infection, indigestion, functional indigestion, Zollinger-Ellison syndrome, non-erosive reflux disease (NERD), visceral-related pain, heartburn, nausea, esophagitis, difficulty swallowing, drooling, airway disorder, or asthma. Furthermore, the pharmaceutical composition of the present invention may effectively prevent, inhibit and treat gastrointestinal disorders such as ulcers and gastrointestinal bleeding, which are side effects caused by administration of clopidogrel.

In the present specification, the delayed release of the first active ingredient may mean that the first active ingredient is not dissolved at acidic pH such as stomach pH, but is released from the pharmaceutical composition at a pH higher than the acidic pH. Specifically, the first active ingredient in the pharmaceutical composition of the present disclosure may start to be released at pH 5 or higher. Therefore, in case that the pharmaceutical composition of the present disclosure is taken, the second active ingredient (clopidogrel) may be released first, and the first active ingredient (tegoprazan) may be released later. Specifically, in case that the pharmaceutical composition of the present disclosure is taken, 80% or more of the second active ingredient (clopidogrel) may be dissolved within 2 hours, but the first active ingredient (tegoprazan) may be released from about 2 hours, and 10% or less thereof may be released within 2 hours.

In the present specification, tegoprazan is a compound represented by the following Formula 1, and has a chemical name of (S)-4-((5,7-difluorochroman-4-yl)oxy)-N,N,2-trimethyl-1H-benzo[d]imidazole-6-carboxamide.

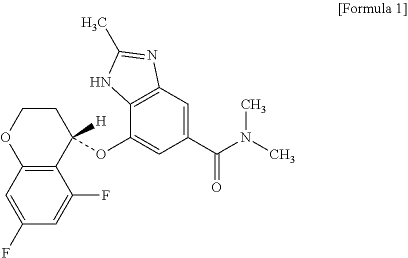 [SMILES: Cc1nc2cc(C(=O)N(C)C)cc(O[C@H]3CCOc4cc(F)cc(F)c43)c2[nH]1]

In the present specification, the term “tegoprazan” may refer to tegoprazan, an optical isomer thereof, a pharmaceutically acceptable salt thereof, a hydrate or solvate thereof, or a mixture thereof.

Tegoprazan is an acid secretion inhibitor and may be effectively used for the treatment of diseases mediated by acid pump antagonistic activity, such as gastrointestinal tract disease, gastroesophageal disease, gastroesophageal reflux disease (GERD), peptic ulcer, gastric ulcer, duodenal ulcer, NSAID-induced ulcer, gastritis, Helicobacter pylori infectious disease, indigestion, functional indigestion, Zollinger-Ellison syndrome, non-erosive reflux disease (NERD), visceral-related pain, heartburn, nausea, esophagitis, difficulty swallowing, drooling, airway disorder, or asthma, but diseases for which tegoprazan may be used are not limited to the above-listed diseases.

According to examples of the present disclosure, the pharmaceutically acceptable salts of tegoprazan may include acid addition salts and base addition salts (including disalts). The acid addition salts include, for example, the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate or xinofoate salts, but not limited to. The base addition salt includes, for example, alkali metal salts such as lithium salts, sodium salts and potassium salts; alkaline earth metal salts such as calcium salts and magnesium salts; ammonium salts; and organic base salts such as triethylamine salts, diisopropylamine salts, or cyclohexylamine salts, but not limited to.

In the present disclosure, the clopidogrel is methyl (+)-(S)-α-(2-chlorophenyl)-6,7-dihydrothieno[3,2-c]pyridine-5(4H)-acetate and is specifically represented by the following Formula 2. Clopidogrel is effectively used for the treatment of stroke, thrombosis, embolism, and peripheral or coronary artery diseases such as myocardial infarction.

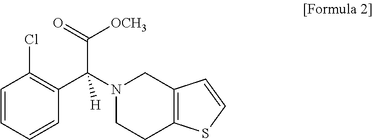 [SMILES: COC(=O)[C@H](c1ccccc1Cl)N1CCc2sccc2C1]

In the present disclosure, the term “clopidogrel” may refer to clopidogrel, an optical isomer thereof, a pharmaceutically acceptable salt thereof, a hydrate or solvate thereof, or a mixture thereof.

According to examples of the present disclosure, the pharmaceutically acceptable salt of clopidogrel may be an acid salt of clopidogrel. Specifically, the pharmaceutically acceptable salt of clopidogrel may be a hydrogen sulfate, a resinate salt, a camsylate salt, a besylate salt, a napadisilate monohydrate salt, a hydrochloride salt, a tartrate salt, an acetate salt, a taurocholate salt, or a mixture thereof. For example, it may be a hydrogen sulfate salt of clopidogrel.

Clopidogrel is a drug that inhibits the aggregation of platelets and exhibits an excellent antithrombotic effect in cardiovascular disease patients. This drug is administered in the form of an inactive prodrug, is activated by the CYP enzymes in vivo, and inhibits platelet aggregation by irreversibly blocking the P2Y12 receptor of platelets. Among the CYP enzymes, especially CYP2C19 is an enzyme that plays the most important role in the activation of clopidogrel, and it was found that, when the function of the CYP2C19 gene is reduced due to a genetic defect thereof, the ability of clopidogrel to inhibit platelet aggregation is lowered, and hence, in patients with this condition, the risk of cardiovascular diseases increase even when clopidogrel is taken. Therefore, the effect of clopidogrel may be relatively low in poor metabolizers associated with a polymorphism in the CYP2C19 gene.

Meanwhile, proton pump inhibitors have inhibitory ability against CYP2C19 and, as a result, competitively inhibit the metabolism of CYP2C19 enzyme when taken with clopidogrel. Thus, the effect of clopidogrel is inferior in poor metabolizers associated with a polymorphism in the CYP2C19 gene, and if the competitive inhibition of the proton pump inhibitor against the CYP2C19 enzyme is added, the effect of clopidogrel may be further lowered. Especially, the proportion of poor metabolizers of the CYP2C19 enzyme is higher in Asians than in Westerners (1 to 4% for Westerners vs. 12 to 23% for Asians).

However, clopidogrel, which is the second active ingredient in the pharmaceutical composition of the present disclosure, may exhibit an excellent therapeutic effect against thrombosis-related diseases by exhibiting sufficient bioavailability, and may effective prevent or treat other gastrointestinal disorders caused by administration of clopidogrel.

In the examples of the present disclosure, the pharmaceutical composition of the present disclosure may further include an additional antiplatelet agent, in addition to the first and second active ingredients. In addition, the pharmaceutical composition of the present disclosure may be administered sequentially or simultaneously with the additional therapeutic agent, and may be administered in single or multiple doses.

In the present specification, the term “prevention” includes preventing, delaying, or inhibiting the development of a disease, and “treatment” includes alleviating disease symptoms, or preventing disease worsening, or delaying, or inhibiting a disease. For example, in the present specification, “preventing or treating the gastrointestinal disorder” includes preventing, delaying or inhibiting the development of gastrointestinal disorders corresponding to side effects caused by administration of clopidogrel, and also includes alleviating symptoms of the gastrointestinal disorder, preventing worsening of the gastrointestinal disorder, or delaying or inhibiting the gastrointestinal disorder.

In the present specification, the term “administration” means providing an active ingredient to a subject by any suitable method, and the pharmaceutical composition of the present disclosure may be administered through any conventional route as long as it may reach the target tissue.

In the present disclosure, the term “subject” refers to mammals, including, but not limited to, humans, guinea pigs, monkeys, cows, horses, sheep, pigs, chickens, turkeys, quails, cats, dogs, mice, mice, or rabbits. Specifically, the subject may be a human being.

In the present disclosure, the term “compartment” may refer to a region including an active ingredient, such as a particle including the active ingredient, a layer including the particle, or a layer including the active ingredient, in the pharmaceutical composition of the present disclosure, and may refer to either a particle itself, a layer including the particle, or a layer including an active ingredient, depending on the unit dosage form of the pharmaceutical composition.

According to examples of the present disclosure, the first compartment containing the first active ingredient and the second compartment containing the second active ingredient, in the pharmaceutical composition, are co-administered, and the pharmaceutical composition may be a composition for co-administration. Specifically, the pharmaceutical composition for co-administration may be a composition that includes the first compartment containing the first active ingredient and the second compartment containing the second active ingredient, or may be a combination that includes the first compartment containing the first active ingredient and the second compartment containing the second active ingredient, or may be a composition including the combination. The composition including the combination may be, for example, a kit. In the present disclosure, the term “composition” may be used interchangeably with the term “combination”.

In examples of the present disclosure, the pharmaceutical composition may be a combination of two ingredients formulated in separate unit dosage forms. In this case, the first active ingredient and the second active ingredient may be co-administered in separate dosage forms.

According to examples of the present disclosure, the first compartment in the pharmaceutical composition (combination) for co-administration may be a unit dosage form containing the first active ingredient, and the second compartment may be a unit dosage form containing the second active ingredient.

According to examples of the present disclosure, the pharmaceutical composition of the present disclosure may be provided in a kit form that includes a unit dosage form containing the first active ingredient and a unit dosage form containing the second active ingredient. The kit may optionally include other elements, such as additional reagents or instructions for use, in addition to the unit dosage forms containing the first active ingredient and the second active ingredient, respectively.

In examples of the present disclosure, in the pharmaceutical composition (combination) for co-administration, the unit dosage form containing the first active ingredient may include an enteric coating layer including an enteric material that is soluble in a pH-dependent manner. Specifically, the enteric coating layer including the pH-dependently soluble enteric material may be dissolved at pH 5 or higher without being dissolved at the acidic pH as stomach pH. For example, the enteric coating layer is insoluble in the pH of the environment such as the stomach, and may be soluble in the environment such as the intestine.

The enteric coating layer is not dissolved at all or is hardly dissolved at less than pH 5 (10% or less, 5% or less, 1% or less), but may be dissolved rapidly at pH 5 or more (pH 5.5, 6, 6.5 or 7). Thus, the first active ingredient is not released at all or is hardly released from the unit dosage form at a pH of less than pH 5 (e.g., in a gastric juice environment), but may be released at a pH of pH 5 or more (e.g., in an intestinal fluid environment), and in case that the pharmaceutical composition is administered to a subject, the first active ingredient may not be released from the pharmaceutical composition in a gastric juice environment, but may be rapidly released in an intestinal fluid environment.

In examples of the present disclosure, the enteric material included in the enteric coating layer may be soluble at pH 5 or higher, specifically at a pH of pH 5 to 7.5, and may be hydroxypropylmethylcellulose phthalate, cellulose acetate phthalate, carboxymethylethyl cellulose, a methacrylic acid-methyl methacrylate copolymer, a methacrylic acid-ethyl acrylate copolymer, an ethyl acrylate-methyl methacrylate trimethylammonium ethyl methacrylate chloride copolymer, a methyl methacrylate-ethyl acrylate copolymer, a methacrylic acid-methylacrylate-methyl methacrylate copolymer, hydroxypropyl cellulose acetate succinate, polyvinyl acetate phthalate, or a mixture thereof. Specifically, the enteric material may be a methacrylic acid-methyl methacrylate copolymer, a methacrylic acid-ethyl acrylate copolymer, or a mixture thereof. More specifically, it may be methacrylic acid-methyl methacrylate copolymer (1:1), methacrylic acid-methyl methacrylate copolymer (1:2), a methacrylic acid-ethyl acrylate copolymer, or a mixture thereof.

In examples of the present disclosure, the enteric coating layer may be prepared with a solution obtained by dissolving the enteric material in an appropriate solvent. In this case, the solvent may be water, an organic solvent, or a mixture thereof. Specifically, the solvent may be water, a C₁ to C₅ linear or branched alcohol, acetone, or a mixture thereof. More specifically, the solvent may be water, methanol, ethanol, isopropyl alcohol, acetone, or a mixture thereof. Even more specifically, the solvent may be water, ethanol, or an aqueous ethanol solution.

In examples of the present disclosure, the solution obtained by dissolving the enteric material in an appropriate solvent may contain triethyl citrate, polysorbate 80, or a mixture thereof.

In examples of the present disclosure, the unit dosage form containing the first active ingredient in the pharmaceutical composition (combination) for co-administration may include a particle including: a core containing the first active ingredient; and an enteric coating layer positioned on the core and surrounding the core.

In examples of the present disclosure, the particle in the pharmaceutical composition (combination) for co-administration may be a tablet, a pellet, or a granule.

In one embodiment, in case that the particle is a tablet, the core may be an uncoated tablet including a granule containing the first active ingredient. The uncoated tablet may further include a pharmaceutically acceptable additive, and the pharmaceutically acceptable additive may be included inside the granule, or outside the granule, or inside and outside the granule. In this case, an enteric coating layer may be formed, which is positioned on the uncoated tablet and surrounds the uncoated tablet.

In another embodiment, in case that the particle is a granule, the core may be an initial granule containing the first active ingredient. The granule may further contain a pharmaceutically acceptable additive. In this case, an enteric coating layer may be formed, which is positioned on the initial granule and surrounds the initial granule may be formed.

In still another embodiment, in case that the particle is a pellet, the core may include a seed and a coating layer positioned on the seed and containing the first active ingredient. The seed may be a sugar seed that does not contain a pharmaceutically active ingredient, and the coating layer containing the first active ingredient may further include a pharmaceutically acceptable additive. In this case, an enteric coating layer may be formed, which is positioned on the pellet and surrounds the pellet.

In examples of the present disclosure, the unit dosage form containing the first active ingredient in the pharmaceutical composition (combination) for co-administration may be a tablet or a capsule.

In examples of the present disclosure, in case that the unit dosage form containing the first active ingredient is a capsule, the capsule may be filled with a particle including: a core containing the first active ingredient; and an enteric coating layer positioned on the core and surrounding the core, wherein the particle may be a tablet, a granule or a pellet. In this case, the capsule may contain a tablet, a granule, a pellet, or a mixture thereof, and specifically, may contain a pellet or a mixture of a pellet and a granule.

In examples of the present disclosure, in case that the unit dosage form containing the first active ingredient is a tablet, the tablet may include: an uncoated core containing the first active ingredient; and an enteric coating layer positioned on the uncoated tablet and surrounding the uncoated tablet. In this case, the unit dosage form may be a multilayered-tablet in the form of tablet-in-tablet (tab-in-tab), and the core which is a uncoated tablet may include: a granule containing the first active ingredient; and a pharmaceutically acceptable additive inside and/or outside the granule, and the uncoated tablet may be prepared by tableting a mixture containing: a granule containing the first active ingredient and a pharmaceutically acceptable additive; and a pharmaceutically acceptable additive besides the granule.

In examples of the present disclosure, in case that the unit dosage form containing the first active ingredient is a tablet, the tablet may include a granule including: a core that is an initial granule containing the first active ingredient; and an enteric coating layer positioned on the initial granule and surrounding the initial granule. In this case, the core which is initial granule may further contain a pharmaceutically acceptable additive, and the tablet may be prepared by tableting a mixture including: a granule containing the first active ingredient and an enteric coating layer; and a pharmaceutically acceptable additive outside the granule.

In examples of the present disclosure, the particle of the unit dosage form containing the first active ingredient may further include at least one additional coating layer. The additional coating layer may be positioned between the core and the enteric coating layer, or positioned on the enteric coating layer, or positioned both between the core and the enteric coating layer and on the enteric coating layer. The additional coating layer may function as an isolation layer that reliably prevents contact with a material that may affect the stability of the first active ingredient, and it may improve the stability of the unit dosage form. In the present disclosure, the term “additional coating layer” may be used interchangeably with the tam “separation layer” or “isolation layer”.

In examples of the present disclosure, the additional coating layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture thereof. Specifically, the additional coating layer may include hydroxypropyl methyl cellulose (HPMC), hydroxypropyl cellulose (HPC), lactose hydrate, microcrystalline cellulose, polyethylene glycol, silicon dioxide, sodium stearyl fumarate, or a mixture thereof.

In examples of the present disclosure, the additional coating layer may be formed by dissolving a pharmaceutically acceptable additive in a solvent and coating the solution on the core which is the first layer. For example, the isolation layer may be prepared with a solution prepared by dissolving hydroxypropyl methylcellulose (HPMC), hydroxypropyl cellulose (HPC), polyethylene glycol, or a mixture thereof in a solvent such as water.

In examples of the present disclosure, the unit dosage form containing the second active ingredient in the pharmaceutical composition (combination) for co-administration may be a particle containing the second active ingredient.

In examples of the present disclosure, the particle containing the second active ingredient in the pharmaceutical composition (combination) for co-administration may be a tablet, a pellet or a granule.

In examples of the present disclosure, the particle containing the second active ingredient in the pharmaceutical composition (combination) for co-administration may be a tablet or a capsule. In one example, in case that the unit dosage form containing the second active ingredient is a capsule, it may be prepared by filling a granule, a tablet, a pellet, or a mixture thereof, which includes the second active ingredient, wherein the granule, tablet or pellet may further include a pharmaceutically acceptable additive. According to another embodiment, in case that the unit dosage form containing the second active ingredient is a tablet, the unit dosage form containing the second active ingredient may be prepared by tableting a mixture including: a granule containing the second active ingredient; and a pharmaceutically acceptable additive. In this case, the granule may further include a pharmaceutically acceptable additive.

In examples of the present disclosure, the pharmaceutical composition may contain the first active ingredient (tegoprazan) in an amount of 5 mg to 100 mg, specifically 10 mg to 60 mg, more specifically 15 mg to 60 mg.

In examples of the present disclosure, the pharmaceutical composition may contain the second active ingredient (clopidogrel) in an amount of 10 to 300 mg, specifically 75 to 300 mg.

In examples of the present disclosure, the pharmaceutical composition (combination) for co-administration may include one or more unit dosage forms containing the first active ingredient and one or more unit dosage forms containing the second active ingredient, and the number of the unit dosage forms in the pharmaceutical composition may be appropriately adjusted depending on the dose of each of the first and second active ingredients and the content of each of the first and second active ingredients in the unit dosage forms.

In examples of the present disclosure, the pharmaceutical composition may be a unit dosage form complex including both the first active ingredient and the second active ingredient. In this case, the first active ingredient (tegoprazan) and the second active ingredient (clopidogrel) may be included together in a single unit dosage form. For example, in case that the unit dosage form is a tablet, the tablet may include both the first active ingredient and the second active ingredient.

In examples of the present disclosure, in the pharmaceutical composition that is a complex, the unit dosage form may include: a first layer which is a first compartment containing the first active ingredient; and a second layer which is a second compartment containing the second active ingredient, wherein the first active ingredient may be delayed-released from the complex, and the second active ingredient may be released immediately. In the present disclosure, the term “first compartment” may be used interchangeably with the term “first layer”, and the term “second compartment” may be used interchangeably with the term “second layer”.

In examples of the present disclosure, in the pharmaceutical composition which is a complex, the unit dosage form may be a tablet or capsule, wherein the tablet may be a multilayered-tablet in a form in which one or more layers are continuously stacked (bi-layer tablet or tri-layer tablet) or multilayered-tablet in the form of tablet-in-tablet (bi-layer tablet or tri-layer tablet).

In the present specification, a multilayered-tablet may be a tablet in which one or more layers surrounding a core are positioned on the core, and the one or more layers may be coating layers and/or matrix layers. For example, the multilayered-tablet may be a tablet-in-tablet as illustrated in FIG. 1A.

Also, in the present specification, a multilayered-tablet may be in a form in which one or more layers are continuously stacked as illustrated in FIG. 1B. For example, the multilayered-tablet may be a bilayer tablet, a trilayer tablet, etc.

In examples of the present disclosure, the first layer may include a particle including: a core containing the first active ingredient; and an enteric coating layer positioned on the core and surrounding the core. The physical properties of the enteric coating layer and the enteric material included in the enteric coating layer are as described above with respect to the pharmaceutical composition for co-administration, unless there are contradictions.

In the examples of the present disclosure, in the pharmaceutical composition, which is a complex, the particle may be a tablet, a pellet, or a granule.

In examples of the present disclosure, in the pharmaceutical composition which is a complex, the particle may include at least one isolation layer for preventing physical contact between the first active ingredient and the second active ingredient, wherein the isolation layer may be positioned between the first layer (first compartment) containing the first active ingredient and the second layer (second compartment) containing the second active ingredient. According to one embodiment, the isolation layer may be positioned between the core containing the first active ingredient and the enteric coating layer, or positioned on the enteric coating layer, or positioned both between the core containing the first active ingredient and the enteric coating layer and on the enteric coating layer.

In examples of the present disclosure, in the pharmaceutical composition which is a complex, the second layer (second compartment) containing the second active ingredient may be formed on the first layer (first compartment). In one embodiment, in case that the isolation layer is positioned only between the core containing the first active ingredient and the enteric coating layer, the second layer containing the second active ingredient may be positioned on the enteric coating layer of the first layer. In another embodiment, in case that the isolation layer is positioned on the enteric coating layer, the second layer containing the second active ingredient may be formed on the isolation layer.

In the present specification, the term “isolation layer” refers to a means for preventing contact between the first active ingredient tegoprazan and the second active ingredient clopidogrel, and the aspects thereof include all means, such as a film, a wall or a coating, which are applicable to an oral dosage form by a person skilled in the art.

In examples of the present disclosure, the isolation layer may be positioned between the first compartment (first layer) containing the first active ingredient and the second compartment (second layer) containing the second active ingredient.

In examples of the present disclosure, the isolation layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the isolation layer may include hydroxypropyl methyl cellulose (HPMC), hydroxypropyl cellulose (HPC), lactose hydrate, microcrystalline cellulose, polyethylene glycol, silicon dioxide, sodium stearyl fumarate, or a mixture thereof.

In examples of the present disclosure, the isolation layer may be included in an amount of 0.1 wt % to 10 wt %, specifically 0.1 wt % to 7 wt %, more specifically 0.1 wt % to 5 wt %, based on the total weight of the complex. When the isolation layer is included in the complex in the above-described amount (wt %), the stability of the complex may be improved, the complex may have excellent stability while having an appropriate size, and the two ingredients may exhibit sufficient dissolution, thus significantly improving the patient's medication compliance.

In examples of the present disclosure, the isolation layer may be formed by mixing a granule formed using a pharmaceutically acceptable additive with a pharmaceutically acceptable additive outside the granule and tableting the mixture.

In examples of the present disclosure, the isolation layer may be prepared by mixing the pharmaceutically acceptable additive with an appropriate solvent to obtain a composition and coating the composition.

In examples of the present disclosure, in the pharmaceutical composition which is a complex, the particle of the first layer may be a tablet. In this case, the pharmaceutical composition which is a complex may be a capsule filled with mini-tablets in the form of multilayered-tablet or multilayered-tablet in the form of a tab-in-tab.

In one embodiment, in case that the complex is a multilayered-tablet, the multilayered-tablet may include: a first layer (first compartment) which is a particle including a core in the form of core tablet containing the first active ingredient and an enteric coating layer formed on the core and surrounding the core; and a second layer (second compartment) positioned on the first layer and surrounding the first layer and containing the second active ingredient. In this case, the core containing the first active ingredient may be prepared by tableting a mixture that contains the granule containing the first active ingredient and a pharmaceutically acceptable additive, and the second layer containing the second active ingredient may be formed either by coating the first layer containing the first active ingredient with a composition containing the second active ingredient, or by tableting a mixture, which contains a granule containing the second active ingredient and a pharmaceutically acceptable additive outside the granule, together with the core tablet. Wherein, the granule may be a wet granule or direct compression granule containing the first active ingredient or the second active ingredient; and a pharmaceutically acceptable additive.

In another embodiment, in case that the complex is a capsule, the capsule may be a capsule filled with mini-tablets having substantially the same structure as the multilayered-tablet.

In examples of the present disclosure, in the pharmaceutical composition which is a complex, the multilayered-tablet may further include at least one isolation layer for preventing contact between the first active ingredient and the second active ingredient. According to one embodiment, the isolation layer in the multilayered-tablet may be positioned between the core in the form of a core tablet and the enteric coating layer. According to another embodiment, the isolation layer in the multilayered-tablet may be positioned between the enteric coating layer and the second layer containing the second active ingredient. According to still another embodiment, the multilayered-tablet may include two isolation layers, wherein the first isolation layer may be positioned between the core in the form of a core tablet and the enteric coating layer, and the second isolation layer may be positioned between the enteric coating layer and the second layer containing the second active ingredient.

In the examples of the present disclosure, the isolation layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the isolation layer may include hydroxypropyl methyl cellulose (HPMC), hydroxypropyl cellulose (HPC), polyethylene glycol, or a mixture thereof.

In the examples of the present disclosure, the content of the isolation layer in the complex is as described above unless there are contradictions.

In examples of the present disclosure, the isolation layer may be formed by mixing the pharmaceutically acceptable additive with an appropriate solvent to obtain a composition and coating the composition.

In examples of the present disclosure, in the pharmaceutical composition which is a complex, the particle of the first layer (first compartment) may be a granule. In this case, the pharmaceutical composition which is a complex may be a tablet or capsule including the granule. Specifically, in case that the particle of the first layer is a granule, the complex may include the granule comprising: a first layer including a core, which is an initial granule containing the first active ingredient, and an enteric coating layer formed on the core and surrounding the core; and a second layer (second compartment) positioned on the first layer and surrounding the first layer and containing the second active ingredient. In this case, the second layer containing the second active ingredient may be formed by coating the first layer with a composition containing the second active ingredient. Wherein, the core, which is the initial granule containing the first active ingredient, may be a wet granule or direct compression granule containing the first active ingredient and a pharmaceutically acceptable additive, and the composition containing the second active ingredient may further include a pharmaceutically acceptable additive.

Wherein, the granule containing the first active ingredient and the second active ingredient may further include at least one isolation layer for preventing contact between the first active ingredient and the second active ingredient. According to one embodiment, the isolation layer may be positioned between the core of the first layer and the enteric coating layer in the granule containing the first active ingredient and the second active ingredient. According to another embodiment, the isolation layer may be positioned between the enteric coating layer and the second layer containing the second active ingredient in the granule containing the first active ingredient and the second active ingredient. According to still another embodiment, the granule containing the first active ingredient and the second active ingredient may include two isolation layers, wherein the first isolation layer may be positioned between the granule which is the first layer and the enteric coating layer, and the second isolation layer may be positioned between the enteric coating layer and the second layer containing the second active ingredient.

In examples of the present disclosure, the isolation layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the isolation layer may include hydroxypropyl methyl cellulose (HPMC), hydroxypropyl cellulose (HPC), polyethylene glycol, or a mixture thereof.

In examples of the present disclosure, the content of the isolation layer in the complex is as described above unless there are contradictions.

In examples of the present disclosure, the isolation layer may be formed by mixing the pharmaceutically acceptable additive with an appropriate solvent to obtain a composition and coating the composition.

In examples of the present disclosure, in case that the particle included in the first layer in the pharmaceutical composition which is a complex is a granule, the pharmaceutical composition which is a complex may be a tablet or capsule containing: a granule containing the first active ingredient and the second active ingredient; and a pharmaceutically acceptable additive. At this time, in case that the pharmaceutical composition which is a complex is a tablet, the tablet may be prepared by tableting a mixture including: a granule containing the first active ingredient and the second active ingredient; and a pharmaceutically acceptable additive. In addition, in case that the pharmaceutical composition which is a complex is a capsule, the capsule may be filled with a granule containing the first active ingredient and the second active ingredient.

In examples of the present disclosure, in the pharmaceutical composition that is a complex, the particle included in the first layer may be a pellet. In this case, the pharmaceutical composition, which is a complex, may be a capsule filled with the pellet. Specifically, in case that the particle of the first layer is a pellet, the complex may be a pellet including: a first layer including a core which is a seed containing the first active ingredient, a first active ingredient-containing layer formed on the seed core and surrounding the core, and an enteric coating layer positioned on the first active ingredient-containing layer and surrounding the first active ingredient-containing layer; and a second layer positioned on the first layer and surrounding the first layer and containing the second active ingredient. In this case, the core seed does not contain an active ingredient and may be, for example, a sugar sphere, and the first active ingredient-containing layer, the enteric coating layer, and the layer containing the second active ingredient may be formed by coating compositions containing the first active ingredient, the enteric material and the second active ingredient, respectively. At this time, the compositions may further contain a pharmaceutically acceptable additive.

In examples of the present disclosure, in case that the particle included in the first layer in the pharmaceutical composition which is a complex is a pellet, the pharmaceutical composition which is a complex may be capsule filled with a pellet containing the first active ingredient and the second active ingredient.

Wherein, the pellet containing the first active ingredient and the second active ingredient may further include at least one isolation layer for preventing contact between the first active ingredient and the second active ingredient. According to one embodiment, the isolation layer may be positioned between the layer containing the first active ingredient and the enteric coating layer in the pellet containing the first active ingredient and the second active ingredient. According to another embodiment, the isolation layer may be positioned between the enteric coating layer and the second layer containing the second active ingredient in the pellet containing the first active ingredient and the second active ingredient. According to still another embodiment, the pellet containing the first active ingredient and the second active ingredient may include two isolation layers, wherein the first isolation layer may be positioned between the layer containing the first active ingredient and the enteric coating layer, and the second isolation layer may be positioned between the enteric coating layer and the second layer containing the second active ingredient.

In the examples of the present disclosure, the isolation layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the isolation may include hydroxypropyl methyl cellulose (HPMC), hydroxypropyl cellulose (HPC), polyethylene glycol, or a mixture thereof.

In the examples of the present disclosure, the content of the isolation layer in the complex is as described above unless there are contradictions.

In examples of the present disclosure, the isolation layer may be formed by mixing the pharmaceutically acceptable additive with an appropriate solvent to obtain a composition and coating the composition.

In the examples of the present disclosure, in case that the pharmaceutical composition which is a complex is a capsule, the capsule may be filled with two or more types of particles selected from the group consisting of a tablet, a granule and a pellet. Specifically, the capsule may be filled with a mixture of a granule and a pellet, wherein the granule and the pellet are as described above with respect to the case that the particle included in the first layer is a granule and the case that the particle included in the first layer is a pellet.

In the examples of the present disclosure, in case that the pharmaceutical composition which is a complex is a capsule, the capsule may be filled with the particle containing the first active ingredient and the particle containing the second active ingredient, wherein the particles may include only one of the first active ingredient and the second active ingredient. In this case, the particle containing the first active ingredient and the particle containing the second active ingredient may be each independently a granule, a pellet or a tablet; or both the particle containing the first active ingredient and the particle containing the second active ingredient may be granules; or both the particle containing the first active ingredient and the particle containing the second active ingredient may be pellets; or both the particle containing the first active ingredient and the particle containing the second active ingredient may be tablets. Alternatively, the particle containing the first active ingredient may be a granule, and the particle containing the second active ingredient may be a pellet; or the particle containing the first active ingredient may be a pellet, and the particle containing the second active ingredient may be a granule. Alternatively, the particle containing the first active ingredient may be a tablet, and the particle containing the second active ingredient may be a pellet; or the particle containing the first active ingredient is a pellet, and the particle containing the second active ingredient may be a tablet. Alternatively, the particle containing the first active ingredient may be a granule, and the particle containing the second active ingredient may be a tablet; or the particle containing the first active ingredient may be a tablet, and the particle containing the second active ingredient may be a granule.

In one embodiment, in case that the particle containing the first active ingredient is a granule, a pellet or a tablet, the granule, pellet or tablet is substantially the same as the above-described granule, pellet and tablet (multilayered-tablet), except that the layer containing the second active ingredient is not formed in the above-described granule, pellet or tablet as above described. In another embodiment, in case that the particle containing the second active ingredient is a granule, the granule containing the second active ingredient may include the second active ingredient and a pharmaceutically acceptable additive. In still another embodiment, in case that the particle containing the second active ingredient is a pellet, the pellet may be prepared by preparing a seed including no active ingredient and coating the seed with a composition containing the second active ingredient and a pharmaceutically acceptable additive. In still another embodiment, in case that the particle containing the second active ingredient is a tablet, the tablet may be prepared by tableting a mixture containing a granule containing the second active ingredient and a pharmaceutically acceptable additive. For example, the capsule may be filled with a granule containing the first active ingredient and a pellet containing the second active ingredient, or may be filled with a tablet containing the first active ingredient and a tablet containing the second active ingredient.

In the examples of the present disclosure, the particle containing the first active ingredient may further include an isolation layer for preventing contact between the first active ingredient and the second active ingredient. The isolation layer of the particle may be substantially the same as described above with respect to the granule, the pellet and the tablet (multilayered-tablet).

The present disclosure provides a complex including: a first compartment including a first active ingredient; a second compartment including a second active ingredient; and an isolation layer preventing contact between the first active ingredient and the second active ingredient, wherein the first active ingredient is tegoprazan, an optical isomer thereof, a pharmaceutically acceptable salt thereof, a hydrate or solvate thereof, or a mixture thereof, and the second active ingredient is clopidogrel, an optical isomer thereof, a pharmaceutically acceptable salt thereof, a hydrate or solvate thereof, or a mixture thereof.

In case that the first active ingredient (tegoprazan) and the second active ingredient (clopidogrel) are formulated in a single dosage form which is a complex, the first active ingredient tegoprazan and the second active ingredient clopidogrel may come into contact with each other, and the stability of the two ingredients may be lowered. For this reason, the two active ingredients may be included in physically separated compartments, respectively, and an isolation layer capable of preventing contact between the first and second active ingredients may be positioned between the two compartments. Thus, the complex including the isolation layer according to the present disclosure has significantly excellent storage stability, because tegoprazan and clopidogrel do not come into physical contact with each other, and the contents of the two ingredients in the dosage form may be maintained at constant levels for a long period of time, and also the contents of impurities in the dosage form may be maintained for a long time at the same levels as those when the dosage form is initially prepared.

In the present specification, the term “isolation layer” refers to a means for preventing contact between the first active ingredient tegoprazan and the second active ingredient clopidogrel, and the aspects thereof include all means, such as a film, a wall and a coating, which are applicable to an oral dosage form by a person skilled in the art.

In examples of the present disclosure, the isolation layer may be positioned between the first compartment containing the first active ingredient and the second compartment containing the second active ingredient.

In the examples of the present disclosure, the isolation layer may include hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the isolation layer may include hydroxypropyl methyl cellulose (HPMC), hydroxypropyl cellulose (HPC), lactose hydrate, microcrystalline cellulose, polyethylene glycol, silicon dioxide, sodium stearyl fumarate, or a mixture thereof.

In examples of the present disclosure, the isolation layer may be formed by mixing a granule formed using a pharmaceutically acceptable additive with a pharmaceutically acceptable additive outside the granule and tableting the mixture.

In examples of the present disclosure, the isolation layer may be formed by mixing the pharmaceutically acceptable additive with an appropriate solvent to obtain a composition and coating the composition.

In the examples of the present disclosure, the content of the isolation layer in the complex is as described above unless there are contradictions.

According to examples of the present disclosure, the pharmaceutically acceptable salt of clopidogrel may be an acid salt of clopidogrel. Specifically, the pharmaceutically acceptable salt of clopidogrel may be a hydrogen sulfate, a resinate salt, a camsylate salt, a besylate salt, a napadisilate monohydrate salt, a hydrochloride salt, a tartrate salt, an acetate salt, a taurocholate salt, or a mixture thereof. For example, it may be a hydrogen sulfate salt of clopidogrel.

In examples of the present disclosure, the complex may include: a first layer (first compartment) containing the first active ingredient; an isolation layer formed on the first layer and preventing contact between the first active ingredient and the second active ingredient; and a second layer (second compartment) formed on the isolation layer and containing the second active ingredient.

In examples of the present disclosure, the complex may be a tablet or a capsule.

In examples of the present disclosure, where the complex is a tablet, the tablet may be a multilayered-tablet in a form in which one or more layers are continuously stacked or a multilayered-tablet in the form of tablet-in-tablet.

In examples of the present disclosure, in case that the complex is a multilayered-tablet, the tablet may include: a first layer (first compartment) including a granule containing the first active ingredient; a second layer including a granule containing the second active ingredient; and an isolation layer including a pharmaceutically acceptable additive.

In this case, the isolation layer is positioned between the first layer and the second layer to prevent physical contact between the first and second active ingredients, and does not contain an active ingredient exhibiting a pharmacological effect, and the pharmaceutically acceptable additive may be hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. Specifically, the pharmaceutically acceptable additive may be hydroxypropyl methyl cellulose (HPMC), hydroxypropyl cellulose (HPC), lactose hydrate, microcrystalline cellulose, polyethylene glycol, silicon dioxide, sodium stearyl fumarate, or a mixture thereof. For example, the isolation layer may include a granule including the pharmaceutically acceptable additive.

In examples of the present disclosure, the first layer may include a pharmaceutically acceptable additive, wherein the pharmaceutically acceptable additive may be included inside a granule containing the first active ingredient and/or outside the granule.

In the examples of the present disclosure, the granule containing the first active ingredient may further include an enteric coating layer positioned on the granule. In this case, the second active ingredient in the multilayered-tablet may be released immediately, and the first active ingredient tegoprazan may be delayed-released. In this case, the physical properties of the enteric coating layer and the enteric material included in the enteric coating layer are as described above with respect to the pharmaceutical composition for co-administration, unless there are contradictions.

In examples of the present disclosure, in case that the complex is a multilayered-tablet, the tablet includes: a core which is a first layer (first compartment) containing the first active ingredient; an isolation layer which is a coating layer positioned on the first layer core and including a pharmaceutically acceptable additive; and a second layer (second compartment) which is a layer positioned on the isolation layer and containing the second active ingredient.

In this case, the isolation layer is positioned between the first layer (first compartment) and the second layer (second compartment) to prevent physical contact between the first active ingredient and the second active ingredient, and the pharmaceutically acceptable additive may be hydroxypropyl methyl cellulose (HPMC), carboxymethyl cellulose (CMC), polyvinyl alcohol, polyethylene glycol, hydroxypropyl cellulose (HPC), lactose hydrate, microcrystalline cellulose, polyethylene glycol, silicon dioxide, sodium stearyl fumarate, sodium stearate, stearic acid, or a mixture of two or more thereof. More specifically, the pharmaceutically acceptable additive may be hydroxypropyl methyl cellulose (HPMC), carboxymethyl cellulose (CMC), polyethylene glycol, or a mixture thereof.

In examples of the present disclosure, the isolation layer may be formed by dissolving the pharmaceutically acceptable additive in a solvent and coating the core, which is the first layer, with the solution. For example, the isolation layer may be formed using a solution prepared by dissolving hydroxypropyl methyl cellulose (HPMC), hydroxypropyl cellulose (HPC), polyethylene glycol, or a mixture of thereof in a solvent such as water.

In examples of the present disclosure, the core may be in the form of a core tablet. In one embodiment, the core tablet may include a pharmaceutically acceptable additive inside a granule and/or outside the granule. In this case, the first layer, which is the core in the form of a core tablet, may be prepared by tableting a mixture containing a granule containing the first active ingredient and the pharmaceutically acceptable additive.

In examples of the present disclosure, the multilayered-tablet may further include an enteric coating layer. In one embodiment, the enteric coating layer may be positioned between the first layer containing the first active ingredient and the isolation layer or between the isolation layer and the second layer containing the second active ingredient. Specifically, the enteric coating layer may be positioned between the isolation layer and the second layer containing the second active ingredient. In case that the multilayered-tablet includes the enteric coating layer, the second active ingredient clopidogrel in the multilayered-tablet may be released immediately, and the first active ingredient tegoprazan may be delayed-released. In this case, the physical properties of the enteric coating layer and the enteric material included in the enteric coating layer are the same as described above with respect to the pharmaceutical composition for co-administration, unless there are contradictions.

In examples of the present disclosure, the second layer containing the second active ingredient may be formed on the first layer by tableting a mixture, which contains a granule containing the second active ingredient and a pharmaceutically acceptable additive and a pharmaceutically acceptable additive outside the granule, together with a core tablet, or may be formed by coating the first layer with a composition including a second active ingredient and a pharmaceutically acceptable additive.

In examples of the present disclosure, the granule containing the first active ingredient and the granule containing the second active ingredient may be wet granules or direct compression granules.

In the embodiments of the present disclosure, in case that the multilayered-tablet further includes an enteric coating layer, the multilayered-tablet may further include at least one isolation layer. In this case, the first isolation layer may be formed on the first layer and the enteric coating layer, and the second isolation layer may be positioned between the enteric coating layer and the second layer.

In examples of the present disclosure, the complex may be a capsule filled with a granule, a pellet, a tablet, or mixtures thereof.

In examples of the present disclosure, in case that the complex is a capsule comprising granule, the granule may include: a first layer as a core which is an initial granule containing the first active ingredient; an isolation layer formed on the initial granule and surrounding the initial granule; and a second layer containing the second active ingredient. In this case, the isolation layer may be formed by coating the first layer with a composition containing the pharmaceutically acceptable additive, and the second layer containing the second active ingredient may be a coating layer formed by coating the isolation layer with a composition containing the second active ingredient, or may be formed by tableting a mixture that contains a pharmaceutically acceptable additive and a granule containing the second active ingredient. Wherein, the core, which is the initial granule containing the first active ingredient, may be a wet granule or direct compression granule containing the first active ingredient and the pharmaceutically acceptable additive, and the composition containing the second active ingredient may further include a pharmaceutically acceptable additive. In case that, the coating layer containing the second active ingredient and the isolation layer including the pharmaceutically acceptable additive may be each formed by coating a composition prepared by dissolving, in a solvent, the ingredient to be included in each of the coating layer and the isolation layer. In this case, the function of the isolation layer and the type of pharmaceutically acceptable additive included in the isolation layer are the same as described above with respect to the multilayered-tablet, unless there are contradictions.

In examples of the present disclosure, the granule containing the first active ingredient and the second active ingredient may further include an enteric coating layer. In one embodiment, the enteric coating layer may be formed between the first layer, which is a core containing the first active ingredient, and the isolation layer, or may be positioned between the isolation layer and the second layer containing the second active ingredient. Specifically, the enteric coating layer may be positioned between the isolation layer and the second layer containing the second active ingredient.

In the examples of the present disclosure, in case that the complex is a capsule comprising pellet, the pellet includes: a first layer which is a core containing the first active ingredient; an isolation layer positioned on the core and including a pharmaceutically acceptable additive; and a second layer positioned on the isolation layer and containing the second active ingredient.

In examples of the present disclosure, the core may include a seed and a coating layer positioned on the seed and containing the first active ingredient. The seed may be a sugar seed that does not contain a pharmaceutically active ingredient, and the coating layer containing the first active ingredient may further include a pharmaceutically acceptable additive. Wherein, the coating layer containing the first active ingredient, the coating layer containing the second active ingredient, and the isolation layer including a pharmaceutically acceptable additive may be formed by coating each composition prepared by dissolving, in a solvent, the ingredient to be included in each of the coating layers and the isolation layer. At this time, the function of the isolation layer and the type of pharmaceutically acceptable additive included in the isolation layer are the same as described above with respect to the multilayered-tablet, unless there are contradictions.

In examples of the present disclosure, the pellet may further include an enteric coating layer. In one embodiment, the enteric coating layer may be positioned between the first layer, which is a core containing the first active ingredient, and the isolation layer, or may be positioned between the isolation layer and the second layer containing the second active ingredient. Specifically, the enteric coating layer may be positioned between the isolation layer and the second layer containing the second active ingredient.

In examples of the present disclosure, in case that the complex is a capsule, the tablet may be a mini-tablet having substantially the same structure as that of the above-described multilayered-tablet.

In examples of the present disclosure, the mini-tablet containing the first active ingredient and the second active ingredient may further include an enteric coating layer. In one embodiment, the enteric coating layer may be positioned between the first layer, which is a core containing the first active ingredient, and the isolation layer, or may be positioned between the isolation layer and the second layer containing the second active ingredient. Specifically, the enteric coating layer may be positioned between the isolation layer and the second layer containing the second active ingredient.

In the examples of the present disclosure, in case that the complex is a capsule, the capsule may be filled with a particle containing the first active ingredient and a particle containing the second active ingredient, wherein the particles may include only one of the first active ingredient and the second active ingredient. In this case, the particle containing the first active ingredient and the particle containing the second active ingredient may be each independently a granule, a pellet or a tablet; or both the particle containing the first active ingredient and the particle containing the second active ingredient may be granules; or both the particle containing the first active ingredient and the particle containing the second active ingredient may be pellets; or both the particle containing the first active ingredient and the particle containing the second active ingredient may be tablets. Alternatively, the particle containing the first active ingredient may be a granule, and the particle containing the second active ingredient may be a pellet; or the particle containing the first active ingredient may be a pellet, and the particle containing the second active ingredient may be a granule. Alternatively, the particle containing the first active ingredient may be a tablet, and the particle containing the second active ingredient may be a pellet; or the particle containing the first active ingredient is a pellet, and the particle containing the second active ingredient may be a tablet. Alternatively, the particle containing the first active ingredient may be a granule, and the particle containing the second active ingredient may be a tablet; or the particle containing the first active ingredient may be a tablet, and the particle containing the second active ingredient may be a granule.

In one embodiment, in case that the particle containing the first active ingredient is a granule, a pellet or a tablet, the granule, pellet or tablet is substantially the same as the above-described granule, pellet or tablet, except that the layer containing the second active ingredient is not formed in the above-described granule or pellet. In another embodiment, in case that the particle containing the second active ingredient is a granule, the granule containing the second active ingredient may include the second active ingredient and a pharmaceutically acceptable additive. In still another embodiment, in case that the particle containing the second active ingredient is a pellet, the pellet may be prepared by preparing a seed including no active ingredient and coating the seed with a composition containing the second active ingredient and a pharmaceutically acceptable additive. In still another embodiment, in case that the particle containing the second active ingredient is a tablet, the tablet may be prepared by tableting a mixture containing a granule containing the second active ingredient and a pharmaceutically acceptable additive. For example, the capsule may be filled with a granule containing the first active ingredient and a pellet containing the second active ingredient, or may be filled with a tablet containing the first active ingredient and a tablet containing the second active ingredient.

At this time, the granule, pellet and/or tablet containing the first active ingredient may further include an enteric coating layer. In this case, the second active ingredient clopidogrel in the capsule may be released immediately, and the first active ingredient tegoprazan may be delayed-released. In this case, the physical properties of the enteric coating layer and the enteric material included in the enteric coating layer are the same as described above with respect to the pharmaceutical composition for co-administration, unless there are contradictions.

In the present specification, the term “pharmaceutically acceptable additive” may refer to ingredients that do not impair the effects of the active ingredients. Examples of the additive include any additives that are commonly used in each dosage form and pharmaceutically acceptable, for example, fillers, disintegrants, binders, plasticizers, lubricants, coating agents (damp-proof or enteric), pH adjusters, diluents, lubricants, preservatives, buffers, sweeteners, wetting agents, suspending agents, coloring agents, fragrances, excipients, and enteric agents.

In examples of the present disclosure, the granules, tablets or pellets containing the first active ingredient or the second active ingredient or the first and second active ingredients may each independently include a pharmaceutically acceptable filler, disintegrant, binding agent, plasticizer, lubricant, coating agent, pH adjusting agent, or mixture thereof.

In examples of the present disclosure, the filler may be, but is not limited to, microcrystalline cellulose, methyl cellulose, sodium carboxymethyl cellulose, hydroxypropyl methyl cellulose, propylene glycol, lactose, white sugar, glucose, fructose, dextrin, mannitol, sodium alginate, corn starch, potato starch, pregelatinized starch, hydroxypropyl starch, precipitated calcium carbonate, synthetic aluminum silicate, calcium hydrogen phosphate, calcium sulfate, sodium chloride, sodium hydrogen carbonate, purified lanolin, kaolin, urea, colloidal silica gel, casein, primojel, or a mixture thereof. Specifically, the filler in the compartment containing the second active ingredient may be microcrystalline cellulose, methylcellulose, lactose, dextrin, sodium carboxymethylcellulose, mannitol, white sugar, corn starch, pregelatinized starch, precipitated calcium carbonate, calcium hydrogen phosphate, or a mixture thereof.

In the examples of the present disclosure, the disintegrant may be, but is not limited to, guar gum, xanthan gum, sodium starch glycolate, low-substituted hydroxypropyl cellulose, croscarmellose sodium, microcrystalline cellulose, cross-linked polyvinylpyrrolidone, corn starch, gelled starch, dextran, mannitol, sodium or calcium carboxymethyl cellulose, sodium alginate or alginic acid, magnesium aluminum silicate, silicic anhydride, bentonite, montmorillonite, bee gum, sodium bicarbonate, citric acid, carboxymethyl cellulose, cross-linked polyvinylpyrrolidone, pregelatinized starch, or a mixture thereof. Specifically, the disintegrant in the compartment containing the first active ingredient may be sodium starch glycolate, corn starch, bentonite, guar gum, xanthan gum, sodium alginate or alginic acid, low-substituted hydroxypropyl cellulose, microcrystalline cellulose, mannitol, magnesium aluminum silicate, croscarmellose sodium (for example, Ac-Di-Sol®), crosslinked polyvinylpyrrolidone, or a mixture of two or more thereof, and the disintegrant in the compartment containing the second active ingredient may be sodium starch glycolate, croscarmellose sodium, hydroxypropyl cellulose, carboxymethyl cellulose, cross-linked polyvinylpyrrolidone, low-substituted hydroxypropyl cellulose, corn starch, pregelatinized starch, or a mixture.

In examples of the present disclosure, the binding agent may be, but is not limited to, povidone, alginic acid, sodium alginate, carbomer, copovidone, starch, pregelatinized starch, polyethylene glycol, a polyvinylpyrrolidone copolymer, a polyvinyl derivative, microcrystalline cellulose, methyl cellulose, ethyl cellulose, hydroxyethyl cellulose, hydroxypropyl cellulose, hydroxypropyl methyl cellulose, carboxymethyl cellulose and salts thereof, gelatin, gum arabic, sodium caseinate, dextrin, mannitol, lactose, xanthan gum, colloidal silicon dioxide, or a mixture thereof. Specifically, the binding agent in the first compartment containing the first active ingredient may be xanthan gum, sodium alginate, gelatin, gum arabic, dextrin, starch, mannitol, lactose, microcrystalline cellulose, colloidal silicon dioxide, polyethylene glycol, a polyvinylpyrrolidone copolymer, hydroxypropyl cellulose, hydroxypropyl methyl cellulose, or a mixture thereof, and the binding agent in the second compartment containing the second active ingredient may be alginic acid, carbomer, copovidone, starch, pregelatinized starch, polyvinyl derivative, methylcellulose, ethylcellulose, hydroxyethyl cellulose, hydroxypropyl cellulose, hydroxypropyl methyl cellulose, carboxymethyl cellulose and salts thereof, gelatin, gum Arabic, sodium caseinate, or a mixture thereof.

In examplets of the present disclosure, the lubricant may be talc, stearic acid and a salt thereof (e.g., calcium stearate, magnesium stearate, or zinc stearate), sodium stearyl fumarate, silicon dioxide, glyceryl monostearate, polyethylene glycol, sodium benzoate, sodium stearyl fumarate, glyceryl monorate, glyceryl monostearate, glyceryl behenate, glyceryl palmitostearate, paraffin, or a mixture thereof. Specifically, the lubricant in the first compartment containing the first active ingredient may be stearic acid, calcium stearate, magnesium stearate, sodium benzoate, sodium stearyl fumarate, glyceryl monorate, glyceryl monostearate, glyceryl behenate, glyceryl palmitostearate, zinc stearate, paraffin, or a mixture thereof, and the lubricant in the compartment containing the second active ingredient may be talc, stearic acid or a salt thereof, sodium stearyl fumarate, silicon dioxide, glyceryl monostearate, polyethylene glycol, or a mixture thereof.

In the examples of the present disclosure, the plasticizer may be one selected from among glycols, esters, acetyl silicone oil, triethyl citrate, glycerin, glycerin derivatives, or mixtures thereof.

In the examples of the present disclosure, the coating agent may be one selected from among methylcellulose, ethylcellulose, methylhydroxy ethyl cellulose, sodium carboxymethyl cellulose, hydroxypropyl methyl cellulose, hydroxypropyl cellulose, hydroxypropyl methyl cellulose phthalate, hydroxyethyl cellulose, cellulose gum, cellulose acetate butyrate, nitrocellulose, salts thereof, or mixtures thereof.

In the examples of the present disclosure, the pH adjusting agent includes an organic acid, wherein the organic acid may be citric acid, hydrochloric acid, lactic acid, phosphoric acid, propionic acid, sulfuric acid, tartaric acid, fumaric acid, malic acid, or a mixture thereof. Specifically, the pH adjusting agent may be citric acid, tartaric acid, fumaric acid, lactic acid, phosphoric acid, malic acid, or a mixture thereof.

In the examples of the present disclosure, the diluent may be starch, lactose, white sugar, dextrin, dextrose, microcrystalline cellulose, sodium carboxymethyl cellulose, mannitol, sorbitol, xylitol, isomalt, sucrose, calcium hydrogen phosphate, colloidal silicon dioxide, or a mixture thereof. Specifically, the diluent may be microcrystalline cellulose, starch, dextrin, lactose, sucrose, mannitol, xylitol, isomalt, sorbitol, or a mixture thereof.

In examples of the present disclosure, the binding agent and the coating agent may be one or a mixture of two or more selected from the group consisting of sodium carboxymethyl cellulose, ethyl cellulose, hydroxypropyl methyl cellulose, hydroxypropyl cellulose, hydroxyethyl cellulose, polyvinylpyrrolidone, xanthan gum, sodium alginate, and gelatin.

The scope of the present disclosure is not limited to the use of the additives, and the additives may be contained in amounts within conventional ranges depending on the choice of a person skilled in the art.

In examples of the present disclosure, the pharmaceutical compositions of the present disclosure may be compositions for preventing or treating thrombosis-related diseases. Specifically, the pharmaceutical compositions may be compositions for preventing or treating atherosclerotic symptom.

In examples of the present disclosure, the pharmaceutical compositions of the present disclosure may be compositions for preventing or treating gastrointestinal disorders. Specifically, the gastrointestinal disorders may be gastrointestinal disorders caused by administration of clopidogrel, an optical isomer thereof, a pharmaceutically acceptable salt thereof, a hydrate or solvate thereof, or a mixture thereof.

In examples of the present disclosure, the pharmaceutical compositions of the present disclosure may be compositions for preventing or treating thrombosis-related diseases and for preventing or treating gastrointestinal disorders caused by administration of clopidogrel, an optical isomer thereof, a pharmaceutically acceptable salt thereof, a hydrate or solvate thereof, or a mixture thereof. Specifically, the gastrointestinal disorders may be gastrointestinal disorders caused by administration of clopidogrel, an optical isomer thereof, a pharmaceutically acceptable salt thereof, a hydrate or solvate thereof, or a mixture thereof.

The present disclosure also provides a method for preventing or treating thrombosis-related diseases including administering a pharmaceutically effective amount of the pharmaceutical composition of the present disclosure to a subject in need thereof. Specifically, the method may be a method for preventing or treating atherosclerotic symptoms.

In the above-described method, the pharmaceutical composition of the present disclosure is the same as described above, unless there are contradictions. For example, the pharmaceutical composition may be either the above-described pharmaceutical composition which is a complex according to the present disclosure described above or the above-described pharmaceutical composition for co-administration according to the present disclosure. For example, in case that the pharmaceutical composition is a pharmaceutical composition which is a complex, the method may include administering to a subject in need thereof a pharmaceutically effective amount of a unit dosage form containing the first active ingredient and the second active ingredient. For example, in case that the pharmaceutical composition is a pharmaceutical composition for co-administration, the method may include co-administering to a subject in need thereof a pharmaceutically effective amount of a unit dosage form containing the first active ingredient and a pharmaceutically effective amount of a unit dosage form containing the second active ingredient.

The method may also be a method for preventing or treating gastrointestinal disorders. Specifically, the method may be a method for preventing or treating gastrointestinal disorders caused by administration of clopidogrel, an optical isomer thereof, a pharmaceutically acceptable salt thereof, a hydrate or solvate thereof, or a mixture thereof.

In the above methods, the pharmaceutical composition of the present disclosure is the same as descried above, unless there are contradictions. For example, the pharmaceutical composition may be the above-described pharmaceutical composition for co-administration or the above-described pharmaceutical composition which is a complex. For example, in case that the pharmaceutical composition is a pharmaceutical composition which is a complex, the method may include administering to a subject in need thereof a pharmaceutically effective amount of a unit dosage form containing the first active ingredient and the second active ingredient. For example, in case that the pharmaceutical composition is a pharmaceutical composition for co-administration, the method may include co-administering to a subject in need thereof a pharmaceutically effective amount of a unit dosage form containing the first active ingredient and a pharmaceutically effective amount of a unit dosage form containing the second active ingredient. The method may also be a method for preventing or treating thrombosis-related diseases. Specifically, the method may be a method for preventing or treating atherosclerotic symptoms.

In the above-described methods, the contents of the active ingredients in the pharmaceutical composition, the dosage form of the composition, the pharmaceutically acceptable additive, etc. are also the same as described above, unless there are contradictions.

The present disclosure also provides use of the pharmaceutical composition of the present disclosure for preventing or treating thrombosis-related diseases.

The present disclosure also provides use of the pharmaceutical composition of the present disclosure in the manufacture of a medicament for preventing or treating thrombosis-related diseases.

In the above-described uses, the pharmaceutical composition of the present disclosure is the same as described above, unless there are contradictions. For example, the pharmaceutical composition may be the above-described pharmaceutical composition for co-administration or the above-described pharmaceutical composition which is a complex.

In the above-described uses, the thrombosis-related diseases may be the atherosclerotic symptoms.

The above-described uses may also be uses for preventing or treating gastrointestinal disorders. Specifically, the above-described uses may be uses for preventing or treating gastrointestinal disorders caused by administration of clopidogrel, a optical isomer thereof, a pharmaceutically acceptable salt thereof, a hydrate or solvate thereof, or a mixture thereof.

The present disclosure also provides use of the pharmaceutical composition of the present disclosure for preventing or treating gastrointestinal disorders.

The present disclosure also provides use of the pharmaceutical composition of the present disclosure in the manufacture of a medicament for preventing or treating gastrointestinal disorders.

In the above-described uses, the gastrointestinal disorders may be gastrointestinal disorders caused by administration of clopidogrel, an optical isomer thereof, a pharmaceutically acceptable salt thereof, a hydrate or solvate thereof, or a mixture thereof.

In the above-described uses, the pharmaceutical composition of the present disclosure is the same as described above, unless there are contradictions. For example, the pharmaceutical composition may be the above-described pharmaceutical composition for co-administration or the above-described pharmaceutical composition which is a complex.

The above-described uses may also be uses for preventing or treating thrombosis-related diseases. Specifically, the thrombosis-related diseases may be atherosclerotic symptoms.

The present disclosure provides a pharmaceutical composition for preventing or treating gastrointestinal disorders caused by administration of clopidogrel, an optical isomer thereof, a pharmaceutically acceptable salt thereof, a hydrate or solvate thereof, or a mixture thereof, including tegoprazan, an optical isomer thereof, a pharmaceutically acceptable salt thereof, a hydrate or solvate thereof, or a mixture thereof.

The present disclosure also provides a pharmaceutical composition for inhibiting gastric acid secretion, including tegoprazan, an optical isomer thereof, a pharmaceutically acceptable salt thereof, a hydrate or solvate thereof, or a mixture thereof, wherein the pharmaceutical composition is co-administered with clopidogrel, an optical isomer thereof, a pharmaceutically acceptable salt thereof, a hydrate or solvate thereof, or a mixture thereof.

The present disclosure also provides a pharmaceutical combination including: tegoprazan, an optical isomer thereof, a pharmaceutically acceptable salt thereof, a hydrate or solvate thereof, or a mixture thereof; and clopidogrel, an optical isomer thereof, a pharmaceutically acceptable salt thereof, a hydrate or solvate thereof, or a mixture thereof.

The present disclosure also provides a pharmaceutical composition including a pharmaceutical combination: tegoprazan, an optical isomer thereof, a pharmaceutically acceptable salt thereof, a hydrate or solvate thereof, or a mixture thereof; and clopidogrel, an optical isomer thereof, a pharmaceutically acceptable salt thereof, a hydrate or solvate thereof, or a mixture thereof. For example, the pharmaceutical composition including the combination may be a kit.

In examples of the present disclosure, the pharmaceutical compositions (combinations) are compositions (combinations) for preventing or treating thrombosis-related diseases. Specifically, the pharmaceutical compositions (combinations) may be compositions (combinations) for preventing or treating atherosclerotic symptoms. In addition, the pharmaceutical compositions (combinations) may also be pharmaceutical compositions (combinations) for preventing or treating gastrointestinal disorders. In addition, the pharmaceutical compositions (combinations) may be pharmaceutical compositions (combinations) for preventing or treating thrombosis-related diseases and for preventing or treating gastrointestinal disorders. In this case, the gastrointestinal disorders may be gastrointestinal disorders caused by administration of clopidogrel, an optical isomer thereof, a pharmaceutically acceptable salt thereof, a hydrate or solvate thereof, or a mixture thereof.

In the pharmaceutical compositions (combinations), the tegoprazan, optical isomer thereof, pharmaceutically acceptable salt thereof, hydrate or solvate thereof, or mixture thereof may be delayed-released from the pharmaceutical compositions (combinations). In addition, in case that the pharmaceutical compositions (combinations) include clopidogrel, an optical isomer thereof, a pharmaceutically acceptable salt thereof, a hydrate or solvate thereof, or a mixture thereof, the clopidogrel, optical isomer thereof, pharmaceutically acceptable salt thereof, hydrate or solvate thereof, or mixture thereof may be released immediately from the pharmaceutical compositions.

In the pharmaceutical compositions (combinations), the contents of the active ingredients, the dosage forms, the pharmaceutically acceptable additives, etc. are the same as described above, unless there are contradictions.

The present disclosure also provide a method for preventing or treating thrombosis-related diseases in a subject and/or a method for preventing or treating gastrointestinal disorders in a subject, the method including administering to a subject in need thereof a pharmaceutically effective amount of a pharmaceutical composition including, as active ingredients: tegoprazan, an optical isomer thereof, a pharmaceutically acceptable salt thereof, a hydrate or solvate thereof, or a mixture thereof; and clopidogrel, an optical isomer thereof, a pharmaceutically acceptable salt thereof, a hydrate or solvate thereof, or a mixture thereof.

In the method, the tegoprazan, optical isomer thereof, pharmaceutically acceptable salt thereof, hydrate or solvate thereof, or mixture thereof may be delayed-released.

In the method, the thrombosis-related diseases may be atherosclerotic symptoms.

In the method, the gastrointestinal disorders may be gastrointestinal disorders caused by administration of clopidogrel, an optical isomer thereof, a pharmaceutically acceptable salt thereof, a hydrate or solvate thereof, or a mixture thereof.

The present disclosure also provide a method for preventing or treating thrombosis-related diseases in a subject and/or a method for preventing or treating gastrointestinal disorders in a subject, including administering to a subject in need thereof a pharmaceutically effective amount of a pharmaceutical combination including: tegoprazan, an optical isomer thereof, a pharmaceutically acceptable salt thereof, a hydrate or solvate thereof, or a mixture thereof; and clopidogrel, an optical isomer thereof, a pharmaceutically acceptable salt thereof, a hydrate or solvate thereof, or a mixture thereof.

In the method, the tegoprazan, optical isomer thereof, pharmaceutically acceptable salt thereof, hydrate or solvate thereof, or mixture thereof may be delayed-released.

In the method, the thrombosis-related diseases may be atherosclerotic symptoms.

In the method, the gastrointestinal disorders may be gastrointestinal disorders caused by administration of clopidogrel, an optical isomer thereof, a pharmaceutically acceptable salt thereof, a hydrate or solvate thereof, or a mixture thereof.

The present disclosure also provide a method for preventing or treating thrombosis-related diseases in a subject and/or a method for preventing or treating gastrointestinal disorders in a subject, including co-administering to a subject in need thereof a pharmaceutically effective amount of tegoprazan, an optical isomer thereof, a pharmaceutically acceptable salt thereof, a hydrate or solvate thereof, or a mixture thereof, with a pharmaceutically effective amount of clopidogrel, an optical isomer thereof, a pharmaceutically acceptable salt thereof, a hydrate or solvate thereof, or a mixture thereof.

In the method, the tegoprazan, optical isomer thereof, pharmaceutically acceptable salt thereof, hydrate or solvate thereof, or mixture thereof may be delayed-released.

In the method, the thrombosis-related diseases may be atherosclerotic symptoms.

In the method, the gastrointestinal disorders may be gastrointestinal disorders caused by administration of clopidogrel, an optical isomer thereof, a pharmaceutically acceptable salt thereof, a hydrate or solvate thereof, or a mixture thereof.

The present disclosure also provides a method for preventing or treating gastrointestinal disorders caused by administration of clopidogrel, an optical isomer thereof, a pharmaceutically acceptable salt thereof, a hydrate or solvate thereof, or a mixture thereof, including administering to a subject in need thereof a pharmaceutically effective amount of tegoprazan, an optical isomer thereof, a pharmaceutically acceptable salt thereof, a hydrate or solvate thereof, or a mixture thereof.

In the method, the tegoprazan, optical isomer thereof, pharmaceutically acceptable salt thereof, hydrate or solvate thereof, or mixture thereof may be delayed-released.

The present disclosure also provides a method of co-administering a pharmaceutical composition for inhibiting gastric acid secretion including tegoprazan, an optical isomer thereof, a pharmaceutically acceptable salt thereof, a hydrate or solvate thereof, or a mixture thereof, with clopidogrel, an optical isomer thereof, a pharmaceutically acceptable salt thereof, a hydrate or solvate thereof, or a mixture thereof.

In the method, the tegoprazan, optical isomer thereof, pharmaceutically acceptable salt thereof, hydrate or solvate thereof, or mixture thereof may be delayed-released.

In the methods of the present disclosure, the term “pharmaceutically effective amount” refers to an amount sufficient to treat a disease at a reasonable benefit/risk ratio applicable to any medical treatment. The effective dose level of the pharmaceutical composition may be determined depending on factors including the patient's type, the severity of the disease, the activity of the drug, sensitivity to the drug, the duration of administration, the route of administration, excretion rate, the duration of treatment, and drugs used in combination with the composition, as well as other factors well known in the medical field. It is important to administer the composition in the minimum amount that can exhibit the maximum effect without causing side effects, in view of all the above-described factors, and this amount can be easily determined by a person skilled in the art.

Specifically, in the pharmaceutical composition of the present disclosure, the daily dosage of clopidogrel, an optical isomer thereof, a pharmaceutically acceptable salt thereof, a hydrate or solvate thereof, or a mixture thereof may be 10 to 300 mg (as clopidogrel), specifically 75 to 300 mg (as clopidogrel), for an adult. In addition, in the pharmaceutical composition of the present disclosure, the daily dosage of tegoprazan, an optical isomer thereof, a pharmaceutically acceptable salt thereof, a hydrate or solvate thereof, or a mixture thereof may be 10 to 200 mg (as clopidogrel) for an adult, but the scope of the present disclosure is not limited thereto.

In the above-described methods, the pharmaceutical composition, specifically, the content of the active ingredient in the pharmaceutical composition, the dosage form, the pharmaceutically acceptable additive, etc. are the same as described above, unless there are contradictions.

The present disclosure also provides use of a pharmaceutical composition or pharmaceutical combination including, as active ingredients, tegoprazan, an optical isomer thereof, a pharmaceutically acceptable salt thereof, a hydrate or solvate thereof, or a mixture thereof, and clopidogrel, an optical isomer thereof, a pharmaceutically acceptable salt thereof, a hydrate or solvate thereof, or a mixture thereof, for preventing or treating thrombosis-related diseases and/or for preventing or treating gastrointestinal disorders.

The present disclosure also provides use of a pharmaceutical composition or pharmaceutical combination including, as active ingredients, tegoprazan, an optical isomer thereof, a pharmaceutically acceptable salt thereof, a hydrate or solvate thereof, or a mixture thereof, and clopidogrel, an optical isomer thereof, a pharmaceutically acceptable salt thereof, a hydrate or solvate thereof, or a mixture thereof, in the manufacture of a medicament for preventing or treating thrombosis-related diseases and/or for preventing or treating gastrointestinal disorders.

The present disclosure also provides use of a pharmaceutical composition including, as an active ingredient, tegoprazan, an optical isomer thereof, a pharmaceutically acceptable salt thereof, a hydrate or solvate thereof, or a mixture thereof, for treating gastrointestinal disorders.

The present disclosure also provides use of a pharmaceutical composition including, as an active ingredient, tegoprazan, an optical isomer thereof, a pharmaceutically acceptable salt thereof, a hydrate or solvate thereof, or a mixture thereof, in the manufacture of a medicament for treating gastrointestinal disorders.

The pharmaceutical compositions are the same as described above, unless there are contradictions.

In the above-described use, the thrombosis-related diseases may be atherosclerotic symptoms.

In the above-described method, the gastrointestinal disorders may be gastrointestinal disorders caused by administration of clopidogrel, an optical isomer thereof, a pharmaceutically acceptable salt thereof, a hydrate or solvate thereof, or a mixture thereof.

### Advantageous Effects

The present disclosure relates to a pharmaceutical composition that delayed-releases tegoprazan and releases clopidogrel immediately. The pharmaceutical composition may exhibit significantly excellent effects on the prevention and treatment of gastrointestinal disorders caused by administration of clopidogrel and thrombosis-related diseases.

### DESCRIPTION OF DRAWINGS

FIG. 1 illustrates a schematic view of a tablet according to the present disclosure.

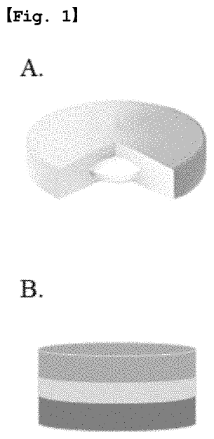

FIG. 2 shows the dissolution rates of clopidogrel from formulations according to the present disclosure.

FIG. 3 shows the dissolution of tegoprazan from formulations according to the present disclosure.

FIG. 4 shows the dissolution of clopidogrel from formulations according to the present disclosure.

FIG. 5 shows the dissolution of tegoprazan from formulations according to the present disclosure.

FIG. 6 shows the dissolution of tegoprazan from formulations according to the present disclosure.

### MODE FOR INVENTION

Hereinafter, the present disclosure will be described in more detail with reference to examples. However, these examples serve to illustrate the present disclosure, and the scope of the present disclosure is not limited by these examples.

### Example 1. Evaluation of Compatibility Between Tegoprazan and Clopidogrel Sulfate

TABLE 1

| 2 weeks of evaluation of | | | | |
|---|---|---|---|---|
| chemical mixture | | | | |
| compatibility | | Initial | | |
| (Packaging condition: | | (room | Accelerated conditions | Stressed conditions |
| HDPE bottle) | | temperature) | (40° C./RH 75 ± 5%) | (60° C./RH 75 ± 5%) |
| Tegoprazan alone | | 99.8% | 100.3% | 99.1% |
| Clopidogrel sulfate alone | | 100.2% | 101.1% | 98.3% |
| Mixture | Tegoprazan | 99.7% | 92.0% | 10.5% |
| thereof | Clopidogrel | 100.3% | 78.9% | 49.0% |
| | sulfate | | | |

According to Table 1 above, in order to examine the effect of each of tegoprazan and clopidogrel hydrogen sulfate, compatibility therebetween was evaluated under storage conditions according to the ICH guideline recommendations and drug safety test regulations.

It was shown that, when the two components came into direct contact with each other under each storage condition, the contents of the two components decreased. This indicates that the stability of the two components is lowered even by physical contact between the two components.

### Example 2: Preparation of Tegoprazan Granules and Tablets

TABLE 2

| Pharmaceutical | Example 2 |
|---|---|
| ingredients | Amount (g)/10,000 tablets (1 batch) |
| Tegoprazan | 250.0 |
| Mannitol | 250.0 |
| Microcrystalline cellulose | 400.0 |
| Croscarmellose sodium | 50.0 |
| Hydroxypropyl cellulose | 30.0 |
| Colloidal silicon dioxide | 10.0 |
| Magnesium stearate | 10.0 |
| Total | 1,000 |

According to Table 2 above, tegoprazan, mannitol, microcrystalline cellulose and croscarmellose sodium were mixed together, sieved through a 750±200 μm mesh, and then placed and mixed in a high-speed mixer. A binding solution was prepared by dissolving hydroxypropyl cellulose in purified water, and the binding solution was introduced into the high-speed mixer, thus preparing wet granules.

The obtained granules were dried while being fluidized in a fluidized bed dryer at an air supply temperature of 60±10° C.

The dried granules were milled using the Quadro Comil on a 750±200-μm mesh, and microcrystalline cellulose, croscarmellose sodium, colloidal silicon dioxide and magnesium stearate were added thereto, mixed, and lubricated to obtain a mixture containing granules of tegoprazan.

The mixture was tableted using an Erweka tablet press (100±20 rpm) to obtain tablets having a rectangular shape.

### Examples 3 and 4: Preparation of Clopidogrel Direct Compression Granules

TABLE 3

| | Example 3 | Example 4 |
|---|---|---|
| Pharmaceutical ingredients | Amount (g)/3,000 tablets (1 batch) | |
| Clopidogrel sulfate | 293.625 | 195.73 |
| Copovidone | — | 24.0 |
| Lactose hydrate | — | 212.7 |
| Anhydrous lactose | 203.0 | — |
| Microcrystalline cellulose | 120.0 | — |
| Pregelatinized starch | 63.0 | — |
| Low-substituted | 36.0 | 50.0 |
| hydroxypropyl cellulose | | |
| Colloidal silicon dioxide | 0.75 | — |
| Light anhydrous silicic acid | — | 5.0 |
| Povidone | 3.6 | — |
| Starch sodium glycolate | 30.0 | — |
| Talc | — | 12.6 |
| Sodium stearyl furmate | 15.0 | 10.0 |
| Total | 764.975 | 510.03 |

According to Table 3 above, clopidogrel sulfate and additives (copovidone, lactose hydrate, anhydrous lactose, microcrystalline cellulose, pregelatinized starch, low-substituted hydroxypropyl cellulose, colloidal silicon dioxide, light anhydrous silicic acid, povidone or sodium starch glycolate) were placed in a mixer (erweka universal) and mixed at 15 rpm for 20 minutes. The mixture was milled using the Quadro Comil on a 750±200 μm mesh to obtain direct compression granules. As a lubricant, sodium stearyl fumurate (Example 3) or talc and sodium stearyl fumurate (Example 4) were added to granules, thus preparing mixtures containing direct compression granules.

### Examples 5 to 14: Preparation of Clopidogrel Granules

TABLE 4

| Pharmaceutical | Example 5 | Example 6 | Example 7 | Example 8 | Example 9 | Example 10 | Example 11 | Example 12 | Example 13 | Example 14 |
|---|---|---|---|---|---|---|---|---|---|---|
| ingredients | Amount (g)/2,000 tablets (1 batch) | | | | | | | | | |
| Clopidogrel | 195.75 | 195.75 | 195.75 | 195.75 | 195.75 | 195.75 | 195.75 | 195.75 | 195.75 | 195.75 |
| sulfate | | | | | | | | | | |
| Microcrystalline | 258.25 | 248.25 | 248.25 | 278.25 | — | — | 252.25 | 252.25 | 252.25 | 252.25 |
| cellulose | | | | | | | | | | |
| Silicified | — | — | — | — | 248.25 | — | — | — | — | — |
| microcrystalline | | | | | | | | | | |
| cellulose | | | | | | | | | | |
| Lactose hydrate | 220.0 | 220.0 | 220.0 | 250.0 | 250.0 | 448.25 | 220.0 | 220.0 | 220.0 | — |
| Mannitol | — | — | — | — | — | — | — | — | — | 220.0 |
| Hyroxypropyl | 50.0 | 50.0 | 50.0 | 50.0 | — | — | 40.0 | 40.0 | 46.0 | 46.0 |
| cellulose | | | | | | | | | | |
| Copovidone | — | — | — | — | 50.0 | 50.0 | — | — | — | — |
| Low-substituted | 56.0 | 60.0 | 36.0 | 36.0 | 36.0 | 80.0 | 36.0 | 36.0 | 36.0 | 36.0 |
| hydroxypropyl | | | | | | | | | | |
| cellulose | | | | | | | | | | |
| Crospovidone | — | — | — | — | — | — | 30.0 | — | — | — |
| Croscarmellose | — | — | 24.0 | 24.0 | — | — | — | 30.0 | 24.0 | 24.0 |
| sodium | | | | | | | | | | |
| Colloidal silicon | — | 10.0 | 10.0 | 10.0 | 10.0 | 10.0 | — | 10.0 | 10.0 | 10.0 |
| dioxide | | | | | | | | | | |
| Light | — | — | — | — | — | — | 10.0 | — | — | — |
| anhydrous | | | | | | | | | | |
| silicic acid | | | | | | | | | | |
| Sodium stearyl | 20.0 | 16.0 | 16.0 | 16.0 | 20.0 | 16.0 | 16.0 | 16.0 | 16.0 | 16.0 |
| fumarate | | | | | | | | | | |
| Total | 800.0 | 800.0 | 800.0 | 860.0 | 810.0 | 800.0 | 800.0 | 800.0 | 800.0 | 800.0 |

### Examples 5 to 8 and Examples 10 to 14

According to Table 4 above, wet granules containing clopidogrel sulfate were prepared.

According to Table 4 above, pharmaceutical additives (microcrystalline cellulose or silicified microcrystalline cellulose, lactose hydrate or mannitol) were mixed together, sieved through a 750±200 μm mesh together with clopidogrel sulfate, and then placed and mixed in a high-speed mixer. According to Table 4 above, a binding solution was prepared by dissolving hydroxypropyl cellulose (Examples 5 to 8 and 11 to 14) or copovidone (Example 10) in purified water (Examples 5 and 6) or anhydrous ethanol (Examples 7 and 8 and 10 to 14), and the binding solution was introduced into the high-speed mixer, thus preparing wet granules.

The obtained granules were dried while being fluidized in a fluidized bed dryer at an air supply temperature of 60±10° C.

The dried granules were milled using the Quadro Comil on a 750±200 μm mesh, and then low-substituted hydroxypropyl cellulose, crospovidone, croscarmellose sodium, light anhydrous silicic acid and/or sodium stearyl fumarate were/was added thereto, mixed, and lubricated to obtain a mixture containing granules.

### Example 9

In Example 9, direct compression granules were prepared using copovidone as a binding agent.

According to Table 4 above, clopidogrel sulfate and additives (excluding the lubricant sodium stearyl fumarate) were placed in a mixer (erweka universal) and then mixed at 15 rpm for 20 minutes. The granules were milled using the Quadro Comil on a 750±200 μm mesh to obtain direct compression granules. As a lubricant, sodium stearyl fumurate were added to the granules, thus preparing a mixture containing direct compression granules.

### Examples 15 and 16: Preparation for Granules for Isolation Between Clopidogrel and Tegoprazan

TABLE 5

| | Example 15 | Example 16 |
|---|---|---|
| Pharmaceutical ingredients | Amount (g)/10,000 tablets (1 batch) | |
| Microcrystalline cellulose | 576.0 | 403.2 |
| Lactose hydrate | — | 172.8 |
| Hydroxypropyl cellulose | 12.0 | 12.0 |
| Silicon dioxide | 6.0 | 6.0 |
| Sodium stearyl fumarate | 6.0 | 6.0 |

According to Table 5 above, granules for forming an isolation layer between the tegoprazan granules and the clopidogrel sulfate were prepared. Specifically, microcrystalline cellulose, lactose hydrate and silicon dioxide were placed and mixed in a high-speed mixer. A binding solution was prepared by dissolving hydroxypropyl cellulose in purified water, and the binding solution was introduced into the high-speed mixer, thus preparing wet granules. The obtained granules were dried while being fluidized in a fluidized bed dryer at an air supply temperature of 60±10° C.

The dried granules were milled using the Quadro Comil on a 750±200 pin mesh, and sodium stearyl fumarate was added as a lubricant to the granules to obtain a mixture containing granules for forming an isolation layer.

### Examples 17 to 19: Preparation of Tegoprazan Wet Granules and Tablets

TABLE 6

| | Example 17 | Example 18 | Example 19 |
|---|---|---|---|
| Pharmaceutical ingredients | Amount (g)/9,408 tablets (1 batch) | | |
| Tegoprazan | 235.21 | 235.21 | 235.21 |
| Mannitol | 235.21 | 235.21 | 235.21 |
| Microcrystalline cellulose | 376.33 | 376.33 | 376.33 |
| Croscarmellose sodium | 47.04 | 47.04 | 47.04 |
| Methacrylic acid-ethyl acrylate | 77.34 | — | — |
| copolymer | | | |
| Methacrylic acid-methyl | — | 77.34 | — |
| methacrylate copolymer (1:1) | | | |
| Methacrylic acid-methyl | — | — | 77.34 |
| methacrylate copolymer (1:2) | | | |
| Triethyl citrate | 10.07 | 10.07 | 10.07 |
| Colloidal silicon dioxide | 9.41 | 9.41 | 9.41 |
| Magnesium stearate | 9.41 | 9.41 | 9.41 |
| Total | 1,000 | 1,000 | 1,000 |

According to Table 6 above, tegoprazan, mannitol, microcrystalline cellulose and croscarmellose sodium were mixed together. The mixture was sieved through a 750±200-μm mesh, and then placed in a high-speed mixer and mixed.

Binding solutions were prepared by each of a methacrylic acid•ethyl acrylate copolymer (Example 17), a methacrylic acid-methyl methacrylate copolymer (1:1) (Example 18), a methacrylic acid-methyl methacrylate copolymer (1:2) (Example 19) and triethyl citrate in 90% ethanol aqueous solution (w/w). Each of the binding solutions was introduced into the high-speed mixer, thus preparing wet granules.

The obtained wet granules were dried while being fluidized in a fluidized bed dryer at an air supply temperature of 60±20° C.

The dried granules were mixed with microcrystalline cellulose, croscarmellose sodium, and colloidal silicon dioxide, and then milled using the Quadro Comil on a 750±200 μm mesh. Then, the granules were mixed and lubricated with magnesium stearate to obtain a mixture containing granules.

The mixture containing granules was tableted using an Erweka tablet press (100±20 rpm) to obtain tablets having a rounded shape.

### Examples 20 to 25: Preparation of Core Tablets and Enteric Tablets Containing Tegoprazan

TABLE 7

| | | Example | Example | Example | Example | Example | Example |
|---|---|---|---|---|---|---|---|
| | Pharmaceutical | 20 | 21 | 22 | 23 | 24 | 25 |
| | ingredients | Amount (g)/10,000 tablets (1 batch) | | | | | |
| Core or | Tegoprazan | 250.0 | 250.0 | 250.0 | 250.0 | 250.0 | 250.0 |
| inner | Mannitol | 250.0 | 250.0 | 250.0 | 250.0 | 250.0 | 250.0 |
| tablet | Microcrystalline | 400.0 | 400.0 | 400.0 | 400.0 | 400.0 | 400.0 |
| | cellulose | | | | | | |
| | Croscarmellose | 50.0 | 50.0 | 50.0 | 50.0 | 50.0 | 50.0 |
| | sodium | | | | | | |
| | Hydroxypropyl | 30.0 | 30.0 | 30.0 | 30.0 | 30.0 | 30.0 |
| | cellulose | | | | | | |
| | Colloidal silicon | 10.0 | 10.0 | 10.0 | 10.0 | 10.0 | 10.0 |
| | dioxide | | | | | | |
| | Magnesium | 10.0 | 10.0 | 10.0 | 10.0 | 10.0 | 10.0 |
| | stearate | | | | | | |
| Total | | 1,000 | 1,000 | 1,000 | 1,000 | 1,000 | 1,000 |
| Weight of tegoprazan core | | 400 | 400 | 400 | 400 | 400 | 400 |
| tablet half-finished product | | | | | | | |
| First | Hypromellose | 12 | 12 | 12 | 12 | 12 | 12 |
| coating | Polyethylene | 1.2 | 1.2 | 1.2 | 1.2 | 1.2 | 1.2 |
| (layer-1) | glycol | | | | | | |
| Second | Methacrylic | 45.44 | — | — | 45.44 | — | — |
| coating | acid•ethyl | | | | | | |
| (layer-2) | acrylate | | | | | | |
| | copolymer | | | | | | |
| | Methacrylic | — | 45.44 | — | — | 45.44 | — |
| | acid-methyl | | | | | | |
| | methacrylate | | | | | | |
| | copolymer (1:1) | | | | | | |
| | Methacrylic | — | — | 45.44 | — | — | 45.44 |
| | acid-methyl | | | | | | |
| | methacrylate | | | | | | |
| | copolymer (1:2) | | | | | | |
| | Triethyl citrate | 5.92 | 5.92 | 5.92 | 5.92 | 5.92 | 5.92 |
| | Polysorbate 80 | 0.16 | 0.16 | 0.16 | 0.16 | 0.16 | 0.16 |
| Third | Hypromellose | 14.0 | 14.0 | 14.0 | — | — | — |
| coating | Polyethylene | 1.28 | 1.28 | 1.28 | — | — | — |
| (layer-3) | glycol | | | | | | |

### Examples 20 to 22

According to Table 7 above, core tablet containing tegoprazan were prepared.

The core tablet containing tegoprazan were prepared using the mixture containing the granules prepared according to Example 2.

The mixture containing the granules prepared according to Example 2 was tableted using a tableting press (erweka universal) equipped with a circular punch having a diameter of 6.5±0.5 mm, thus preparing tablets having a hardness of 12±5 KP.

The prepared tegoprazan core or inner tablets were placed in a coating machine (Freund Industrial) and coated with a solution of hypromellose and polyethylene glycol in purified water to form a first coating layer (layer-1).

After formation of the first coating layer, an enteric coating layer (or a second coating layer) was formed. Specifically, a coating solution was prepared by dissolving a methacrylic acid•ethyl acrylate copolymer (Example 20, solvent: purified water or 90% (w/w) ethanol aqueous solution) or a methacrylic acid-methyl methacrylate copolymer (1:1) (Example 21, solvent: 90% (w/w) ethanol aqueous solution) or a methacrylic acid-methyl methacrylate copolymer (1:2) (Example 22, 90% (w/w) ethanol aqueous solution), triethyl citrate and polysorbate 80 in a suitable solvent (purified water, an organic solvent, or a mixture thereof), and the first coating layer of each of the tegoprazan-containing tablets was coated with the coating solution to form a second coating layer, thus preparing enteric tablets containing tegoprazan according to Table 7 above.

Subsequently, a coating solution was prepared by dissolving hypromellose and polyethylene glycol in purified water, and each of the tegoprazan-containing tablets having the second coating layer formed thereon was coated with the coating solution to form a third coating layer (isolating layer) on the second coating layer.

### Examples 23 to 25

Using the components and contents shown in Table 7 above, tegoprazan-containing enteric tablets, each having a first coating layer and second coating layer formed on a tegoprazan core tablet, were prepared in the same manner as in Examples 20 to 22, except that only the first coating layer and the second coating layer were formed without forming the third coating layer.

In the preparation of Examples 20 to 25, the processes of forming the first to third coating layers were performed in consideration of the air supply/exhaust temperatures, coating pan rotating speed, coating solution supply speed, product temperature, etc. known in a conventional art and to those skilled in the art. The process conditions are summarized in Table 8 below.

TABLE 8

| | Air supply | Air exhaust | | Atom | Pattern | |
|---|---|---|---|---|---|---|
| | temperature | temperature | Pan speed | pressure | pressure | Pump speed |
| First coating | 55 ± 10° C. | 45 ± 10° C. | 5 ± 3 rpm | 1 ± 0.5 bar | 1 ± 0.5 bar | 10 ± 7 rpm |
| Second coating | 60 ± 20° C. | 50 ± 15° C. | 8 ± 5 rpm | 1.5 ± 0.7 bar | 1.5 ± 0.7 bar | 15 ± 9 rpm |
| Third coating | 55 ± 10° C. | 50 ± 15° C. | 7 ± 4 rpm | 1.5 ± 0.7 bar | 1.5 ± 0.7 bar | 15 ± 9 rpm |

### Examples 26 to 28: Preparation of Tegoprazan Core Tablets and Enteric Tablets

TABLE 9

| | Pharmaceutical | Example 26 | Example 27 | Example 28 |
|---|---|---|---|---|
| | ingredients | Amount (g)/10,000 tablets (1 batch) | | |
| Core or inner | Tegoprazan | 250.0 | 250.0 | 250.0 |
| tablet | Mannitol | 250.0 | 250.0 | 250.0 |
| | Microcrystalline | 400.0 | 400.0 | 400.0 |
| | cellulose | | | |
| | Croscarmellose | 50.0 | 50.0 | 50.0 |
| | sodium | | | |
| | Hydroxypropyl | 30.0 | 30.0 | 30.0 |
| | cellulose | | | |
| | Colloidal silicon | 10.0 | 10.0 | 10.0 |
| | dioxide | | | |
| | Magnesium | 10.0 | 10.0 | 10.0 |
| | stearate | | | |
| Total | | 1,000 | 1,000 | 1,000 |
| Weight of tegoprazan core tablet | | 400 | 400 | 400 |
| half-finished product | | | | |
| First coating | Hypromellose | 12 | 12 | 12 |
| (layer-1) | Polyethylene | 1.2 | 1.2 | 1.2 |
| | glycol | | | |
| Second coating | Methacrylic | 53.7 | — | — |
| (layer-2) | acid-ethyl | | | |
| | acrylate | | | |
| | copolymer | | | |
| | Methacrylic | — | 53.7 | — |
| | acid-methyl | | | |
| | methacrylate | | | |
| | copolymer (1:1) | | | |
| | Methacrylic | — | — | 53.7 |
| | acid-methyl | | | |
| | methacrylate | | | |
| | copolymer (1:2) | | | |
| | Triethyl citrate | 6.99 | 6.99 | 6.99 |
| | Polysorbate 80 | 0.19 | 0.19 | 0.19 |
| Third coating | Hypromellose | 16.54 | 16.54 | 16.54 |
| (layer-3) | Polyethylene | 1.51 | 1.51 | 1.51 |
| | glycol | | | |

Tegoprazan-containing tablets of Examples 26 to 28 were prepared in substantially the same manner as in the method of Examples 20 to 22, except that the components and contents shown in Table 9 above were used. At this time, the tegoprazan-containing core tablets were prepared using the mixture containing the granules prepared according to Example 2.

### Examples 29 to 32: Preparation of Multilayered-Tablets Containing Tegoprazan and Clopidogrel

TABLE 10

| Tegoprazan | Example 29 | Example 30 | Example 31 | Example 32 | Weight/tablet |
|---|---|---|---|---|---|
| granules | Granule-containing mixture of Example 2 | | | | 100 mg |
| Granules forisolation | Granule-containing | | Granule-containing | | — |
| | mixture of Example 15 | | mixture of Example 16 | | |
| | 100 mg | 75 mg | 100 mg | 75 mg | — |

| | | | |
|---|---|---|---|
| Clopidogrel sulfate granules | Granule-containing | Granule-containing | 255 mg |
| | mixture of Example 3 | mixture of Example 4 | |

According to Table 10 above, multilayered-tablets (which is in a form in which the layers are continuously stacked) containing tegoprazan and clopidogrel were prepared.

### Examples 29 and 30

Tri-layer tablets were prepared from the granule-containing mixture of Example 2 (granule-containing mixture before tableting), the granule-containing mixture of Example 15 and the granule-containing mixture of Example 3 by means of the ICHIHASHISEIKI Tablet press.

At this time, each tri-layer tablet was prepared in such a manner that the layer formed of the granule-containing mixture of Example 15 was positioned between the tegoprazan-containing layer and the clopidogrel sulfate-containing layer. Tegoprazan and clopidogrel sulfate were isolated from each other by the layer formed of the granule-containing mixture of Example 15.

### Examples 31 and 32

Tri-layer tablets were prepared in the same manner as in Examples 29 and 30, except that the granule-containing mixture of Example 16 and the granule-containing mixture of Example 4 were used. At this time, each tri-layer tablet was prepared in such a manner that the layer formed of the granule-containing mixture of Example 15 was positioned between the tegoprazan-containing layer and the clopidogrel sulfate-containing layer. Tegoprazan and clopidogrel sulfate were isolated from each other by the layer formed of the granule-containing mixture of Example 16.

### Experimental Example 1. Evaluation of Dissolution Tests for Examples 29 to 32

Pharmaceutical equivalence of the tablets according to Examples 29 to 32 and control formulations (Plavix® tablet and K-CAB® tablets) were evaluated by measuring dissolution rate according to the United States Pharmacopeia (USP) apparatus 2 (paddle).

Dissolution conditions were set as follows: pH 2.0 and pH 4.0 (acetate buffer); 37±0.5° C., 900 mL medium; and 50 rpm. The sample solutions obtained after the initiation of dissolution were analyzed using an ultraviolet spectrometer of high-performance liquid chromatography (HPLC; Agilent Technologies) to evaluate the dissolution equivalence of the tablets. The results are shown in Table 11 and 12 below.

TABLE 11
Dissolution rate (%) at pH 2.0

| Sampling time | 5 min | 10 min | 15 min | 30 min | 45 min | 60 min |
|---|---|---|---|---|---|---|
| Plavix ® tablet | 23.10 | 50.84 | 73.17 | 99.61 | 101.20 | 101.68 |
| Example 29 | 22.79 | 50.67 | 76.92 | 103.14 | 103.52 | 103.92 |
| Example 30 | 20.10 | 42.20 | 70.49 | 102.90 | 103.40 | 103.51 |
| Example 31 | 25.86 | 48.41 | 72.37 | 100.38 | 100.69 | 100.83 |
| Example 32 | 27.70 | 54.00 | 73.27 | 99.12 | 99.51 | 99.61 |

TABLE 12
Dissolution rate (%) at pH 4.0

| Sampling time | 5 min | 10 min | 15 min | 30 min | 45 min | 60 min |
|---|---|---|---|---|---|---|
| K-CAB ® tablet | 53.84 | 75.95 | 85.41 | 94.98 | 97.41 | 98.37 |
| Example 29 | 43.14 | 72.77 | 82.30 | 92.56 | 94.78 | 95.43 |
| Example 30 | 36.41 | 58.85 | 69.39 | 93.22 | 98.86 | 99.56 |
| Example 31 | 75.87 | 93.47 | 99.71 | 105.08 | 105.92 | 106.17 |
| Example 32 | 68.07 | 91.31 | 98.36 | 103.05 | 103.09 | 104.13 |

From the dissolution rate measurement result shown in Tables 11 and 12, it could be seen that, even though the tri-layer tablets according to the Examples of the present disclosure were in a single dosage form containing the two active ingredients, each of the ingredients contained in each of the tri-layer tablets showed the same dissolution rate as when each of the ingredients was formulated alone.

Thus, it can be seen that, even though the tablet formulations of the present disclosure were in a single dosage form containing the two active ingredients, they may exhibit the same therapeutic effects as when formulations containing the two ingredients, respectively, are administered alone, thus significantly improving the patient's medication compliance.

### Experimental Example 2. Evaluation of Dissolution Tests for Granule-Containing Tablet Having Enteric Film Formed on Tegoprazan Granules and Tablet Containing Tegoprazan Core Tablet and Having Enteric Coating Layer and Isolation Layer (Evaluation of Dissolution Tests for Examples 2 and 17 to 28)

Pharmaceutical equivalence of the tablets according to Examples 2 and 17 to 28 were evaluated by measuring dissolution rate according to the United States Pharmacopeia (USP) apparatus 2 (paddle), and the results are shown in Table 13 below.

Dissolution conditions were set as follows: pH 1.2; 37±0.5° C.; 900 mL medium; and 50 rpm. The sample solutions obtained after the initiation of dissolution were analyzed using an ultraviolet spectrometer of high-performance liquid chromatography (HPLC; Agilent Technologies) to measure the dissolution rates of the tablets, thereby evaluating the acid resistances of the tablets under an acidic condition.

TABLE 13
Dissolution rate (%) at pH 1.2

| Sampling time | 5 min | 10 min | 15 min | 30 min | 45 min | 60 min | 90 min | 120 min |
|---|---|---|---|---|---|---|---|---|
| Example 2 | 96.52 | 96.81 | 96.84 | 95.84 | 94.47 | 93.47 | 93.40 | 93.11 |
| Example 17 | 0 | 0 | 0 | 0 | 0 | 0 | 0 | 0.8 |
| Example 18 | 0 | 0 | 0 | 0 | 0 | 0 | 0 | 0.2 |
| Example 19 | 0 | 0 | 0 | 0 | 0 | 0 | 0 | 0 |
| Example 20 | 0 | 0 | 0 | 0 | 0 | 0 | 0 | 0 |
| Example 21 | 0 | 0 | 0 | 0 | 0 | 0 | 0 | 0 |
| Example 22 | 0 | 0 | 0 | 0 | 0 | 0 | 0 | 0 |
| Example 23 | 0 | 0 | 0 | 0 | 0 | 0 | 0 | 0 |
| Example 24 | 0 | 0 | 0 | 0 | 0 | 0 | 0 | 0 |
| Example 25 | 0 | 0 | 0 | 0 | 0 | 0 | 0 | 0 |
| Example 26 | 0 | 0 | 0 | 0 | 0 | 0 | 0 | 0 |
| Example 27 | 0 | 0 | 0 | 0 | 0 | 0 | 0 | 0 |
| Example 28 | 0 | 0 | 0 | 0 | 0 | 0 | 0 | 0 |

Referring to Table 13 above, it could be confirmed that, in the case of the tablet that does not contain pharmaceutical additives having enteric properties (Example 2), the tablet showed high dissolution rate in the acidic medium, but in the case of the tegoprazan-containing granules or tablets containing the enteric agent (Examples 7 to 28), the granules or tablets had acid resistance in the acidic medium. In addition, it could be confirmed that the tablets (Examples 26 to 28) including the first coating layer, and the second coating layer (enteric coating layer) formed using the pharmaceutical additives having enteric properties, without having the third coating layer (isolation layer), also had acid resistance.

### Examples 33 to 35: Preparation of Tablet-In-Tablet Formulation Including Tegoprazan Formulation (Including Tegoprazan Core Tablet Having Enteric Coating Layer Formed Thereon) and Clopidogrel Granule

TABLE 14

| Constitution | Tablet-in-tablet | Example 33 | Example 34 | Example 35 | Weight/tablet |
|---|---|---|---|---|---|
| Core or inner | Enteric-coated | Example 20 | Example 21 | Example 22 | 122 mg |
| | formulation including | | | | |
| | tegoprazan core tablet | | | | |
| Shell | Mixture containing | Example 5 | Example 9 | Example 10 | 255 mg |
| | clopidogrel granules | | | | |

According to Table 14 above, tablet-in-tablet formulations were prepared with the mixture of each of Examples 5, 9 and 10 by means of a universal tablet press (ICHIHASHISEIKI tablet press) so that the tegoprazan tablet (Examples 20 to 22) having formed thereon the coating layers including the enteric coating layer was located at the core and so that the clopidogrel-containing granules were located at the shell.

Specifically, the mixture containing the clopidogrel sulfate granules of Example 5, 9 or 10 was placed in the tablet press punch die, and then the coated tegoprazan-containing core tablet of Example 20, 21 or 22 was placed in the punch die. Next, pressure was applied thereto so that the core tablet was not damaged, and tableting was performed, thus preparing tablet-in-tablet formulations.

During the tableting process for preparation of the tablet-in-tablet formulations, the tableting pressure (850 to 1500 kgf) applied to the clopidogrel shell layer did not result in cracking of the tegoprazan core tablet and the enteric coating layer and leaking of the contents therefrom.

### Experimental Example 3. Evaluation of Dissolution Tests for Tablet-In-Tablet Formulations Containing Clopidogrel Sulfate and Tegoprazan (Evaluation of Dissolution Tests for Examples 33 to 35)

Pharmaceutical equivalence of control tablets (Plavix® tablet and K-CAB® tablet) and the tablets according to Examples 33 to 35 was evaluated by measuring dissolution rate according to the United States Pharmacopeia (USP) apparatus 2.

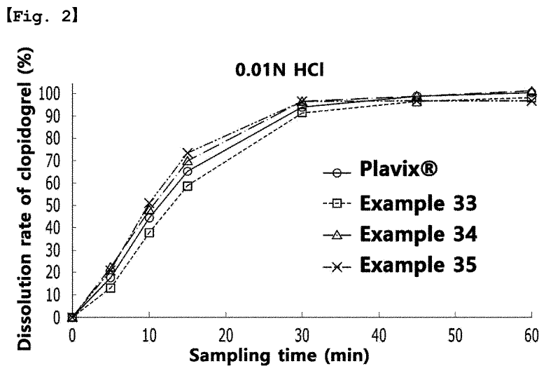

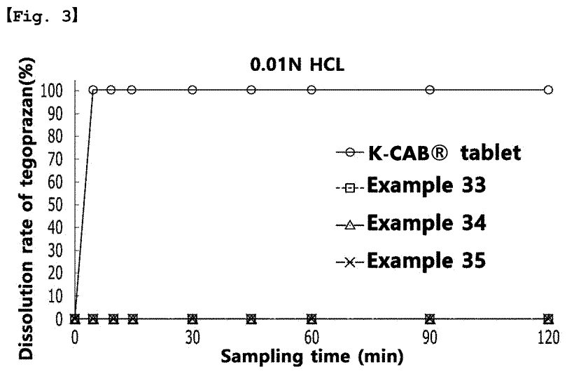

Dissolution conditions were set as follows: 0.01N HCl; 37±0.5° C.; 900 mL medium; and 50 rpm. The sample solutions obtained after the initiation of dissolution were analyzed using an ultraviolet spectrometer of high-performance liquid chromatography (HPLC; Agilent Technologies) to evaluate the dissolution rates of tegoprazan and clopidogrel, and the results are shown in FIGS. 2 and 3.

According to the examination criteria for Pharmaceutical equivalence described in the Korean Pharmacopoeia, where the average dissolution rate of the control tablet (Plavix®) reaches 85% between 15 and 30 minutes, a test tablet can be determined to be equivalent to the control tablet when the value of the similarity factor(f2) is 50 or more or when the average dissolution rate of the test formulation is within ±15% of the average dissolution rate of the control tablet at the time point where the average dissolution rate of the control preparation is around 60% or 85%.

Referring to FIG. 2, it was confirmed that the dissolution rates of the control tablet Plavix® at 15 min and 30 min were 65.4% and 94.7%, respectively, and at this time, the dissolution rates of clopidogrel sulfate from the tablet of Example 33 at 15 min and 30 min were 58.9% and 91.6%, respectively, the dissolution rates of clopidogrel sulfate from the tablet of Example 34 at 15 min and 30 min were 70.4% and 96.8%, respectively, and the dissolution rates of clopidogrel sulfate from the tablet of Example 35 at 15 min and 30 min were 73.7% and 97.0%, respectively.

On the other hand, as shown in FIG. 3, as a result of conducting 2-hour dissolution tests for the tablet-in-tablet formulations of Examples 33 to 35 having the tegoprazan core tablets including the enteric coating layer under a 0.01N HCl medium condition (apparatus 2, 50 rpm), it could be confirmed that tegoprazan in the formulations was hardly dissolved, unlike that in K-CAB® tablet, suggesting that the formulations had acid resistance.

Thus, from the results in FIG. 3, it can be seen that tegoprazan in the tablet-in-tablet formulations of Examples 33 to 35 is not dissolved in the stomach, unlike that the commercially available control tablet K-CAB® tablet, suggesting that tegoprazan is delayed-released from the tablet-in-tablet formulations.

In the case of the tablets according to Examples 33 to 35 of the present disclosure, tegoprazan is not dissolved in the gastric juice condition due to the enteric coating layer, and only clopidogrel is selectively dissolved in the gastric juice condition, and thus the dissolution rate of clopidogrel from the complex including tegoprazan and clopidogrel is maintained at a high level, and as a result, clopidogrel may exhibit significantly excellent efficacy.

### Examples 36 to 42: Preparation of Core Tablets and Enteric Tablets Containing Tegoprazan

TABLE 15

| | | Example | Example | Example | Example | Example | Example | Example |
|---|---|---|---|---|---|---|---|---|
| | Pharmaceutical | 36 | 37 | 38 | 39 | 40 | 41 | 42 |
| | ingredients | Amount (g)/10,000 tablets (1 batch) | | | | | | |
| Core or | Tegoprazan | 250.0 | 250.0 | 250.0 | 250.0 | 250.0 | 250.0 | 500.0 |
| inner | Mannitol | 250.0 | 250.0 | 250.0 | 250.0 | 250.0 | 250.0 | 200.0 |
| tablet | Microcrystalline | 400.0 | 400.0 | 400.0 | 400.0 | 400.0 | 400.0 | 190.0 |
| | cellulose | | | | | | | |
| | Croscarmellose | 50.0 | 50.0 | 50.0 | 50.0 | 50.0 | 50.0 | 50.0 |
| | sodium | | | | | | | |
| | Hydroxypropyl | 30.0 | 30.0 | 30.0 | 30.0 | 30.0 | 30.0 | 30.0 |
| | cellulose | | | | | | | |
| | Colloidal silicon | 10.0 | 10.0 | 10.0 | 10.0 | 10.0 | 10.0 | 20.0 |
| | dioxide | | | | | | | |
| | Magnesium | 10.0 | 10.0 | 10.0 | 10.0 | 10.0 | 10.0 | 10.0 |
| | stearate | | | | | | | |
| Total | | 1000.0 | 1000.0 | 1000.0 | 1000.0 | 1000.0 | 1000.0 | 1000.0 |
| Weight of tegoprazan core | | 400.0 | 400.0 | 400.0 | 400.0 | 400.0 | 400.0 | 400.0 |
| tablet half-finished product | | | | | | | | |
| First | Hypromellose | 8.0 | 8.0 | 8.0 | 8.0 | 8.0 | 8.0 | 8.0 |
| coating | Polyethylene | 0.8 | 0.8 | 0.8 | 0.8 | 0.8 | 0.8 | 0.8 |
| (Layer-1) | glycol | | | | | | | |
| Second | Hypromellose | 61.32 | — | — | — | — | — | — |
| coating | phthalate | | | | | | | |
| (Layer-2) | Methacrylic | — | 40.87 | — | — | — | — | — |
| | acid•ethyl | | | | | | | |
| | acrylate | | | | | | | |
| | copolymer | | | | | | | |
| | Methacrylic | — | — | 49.06 | 40.88 | 30.66 | 20.44 | 20.44 |
| | acid•methyl | | | | | | | |
| | methacrylate | | | | | | | |
| | copolymer (1:1) | | | | | | | |
| | Methacrylic | — | — | 12.26 | 20.44 | 30.66 | 40.88 | 30.66 |
| | acid•methyl | | | | | | | |
| | methacrylate | | | | | | | |
| | copolymer (1:2) | | | | | | | |
| | Triethyl citrate | 4.91 | 6.40 | 7.96 | 7.96 | 7.96 | 7.96 | 7.96 |
| | Polysorbate 80 | — | 0.16 | 0.20 | 0.20 | 0.20 | 0.20 | 0.20 |
| | Talc | 6.13 | — | 6.12 | 6.12 | 6.12 | 6.12 | 6.12 |
| Third | Hypromellose | 14.32 | 14.32 | 14.32 | 14.32 | 14.32 | 14.32 | 14.32 |
| coating | Polyethylene | 1.36 | 1.36 | 1.36 | 1.36 | 1.36 | 1.36 | 1.36 |
| (Layer-3) | glycol | | | | | | | |

The tegoprazan-containing tablets of Examples 36 to 42 were prepared in substantially the same manner as in Examples 20 to 22, except that the components and contents shown in Table 15 above were used.

For preparation of the tegoprazan-containing core tablets, the core tablets of Examples 36 to 41 were prepared with the granule-containing mixture prepared according to Example 2, and the tegoprazan core tablet of Example 42 was prepared with a mixture prepared in the same manner as in Example 2 using the components and contents shown in Table 15 above.

### Examples 43 to 48: Preparation of Tablet-Tablet Formulation Containing Tegoprazan Enteric Tablet and Clopidogrel Granule

TABLE 16

| Constitution | Tablet-in-tablet | Example | Example | Example | Example | Example | Example |
|---|---|---|---|---|---|---|---|
| | | 43 | 44 | 45 | 46 | 47 | 48 |
| Core or | Tegoprazan | Example | Example | Example | Example | Example | Example |
| inner | enteric tablet | 36 | 37 | 38 | 39 | 40 | 41 |

| | | |
|---|---|---|
| Shell | Clopidogrel | Example 7 |
| | granule- | |
| | containing | |
| | mixture | |

According to the combinations shown in Table 16 above, tablet-in-tablet formulations, each containing a tegoprazan enteric tablet and a clopidogrel granule, were prepared in substantially the same manner as the preparation of the tablet-in-tablet formulations according to the method of Examples 33 to 35.

During preparation of the tablet-in-tablet formulations of Examples 43 to 48, various tableting pressures (850 to 1500 kgf) did not result in cracking of the tegoprazan core tablet and the enteric coating layer.

### Experimental Example 4. Evaluation of Tablet-in-Tablet Formulation Containing Clopidogrel Sulfate and Tegoprazan

### Experimental Example 4-1. Evaluation of Stability of Tegoprazan Enteric Tablet and Tablet-in-Tablet Formulation

TABLE 17

| | | Example 41 | | Example 42 | |
|---|---|---|---|---|---|
| | | | 1 month under | | 1 month under |
| | | | stressed | | stressed |
| (Packaging | | | conditions | | conditions |
| condition: | | | (60° C./ | | 60 °C./ |
| HDPE bottle) | | Initial | RH75 ± 5%) | Initial | RH75 ± 5%) |
| Purity | Individual | 0.06% | 0.05% | 0.04% | 0.04% |
| | maximum | | | | |
| | Total | 0.14% | 0.13% | 0.08% | 0.08% |

TABLE 18

| | | | Example | Example | Example | Example |
|---|---|---|---|---|---|---|
| (Packaging condition: HDPE bottle) | | | 43 | 44 | 46 | 48 |
| Initial | Tegoprazan | Individual | 0.05% | 0.05% | 0.04% | 0.06% |
| | | maximum | | | | |
| | | Total | 0.10% | 0.19% | 0.09% | 0.14% |
| | Clopidogrel | Impurity 1 | 0.07% | 0.07% | 0.05% | 0.06% |
| | | Impurity 2 | 0.02% | 0.02% | 0.02% | 0.03% |
| | | Total | 0.19% | 0.20% | 0.17% | 0.21% |
| 1 month under | Tegoprazan | Individual | 0.09% | 0.08% | 0.07% | N/D |
| accelerated | | maximum | | | | |
| conditions | | Total | 0.13% | 0.21% | 0.11% | N/D |
| (40° C./RH75 ± 5%) | Clopidogrel | Impurity 1 | 0.18% | 0.17% | 0.18% | 0.17% |
| | | Impurity 2 | 0.07% | 0.07% | 0.08% | 0.07% |
| | | Total | 0.39% | 0.39% | 0.40% | 0.36% |

(N/D means that no impurities were detected.)

For stability tests for the tegoprazan enteric tablets of Examples 41 and 42 and the tablet-in-tablet formulations of Examples 43, 44, 46 and 48, evaluation was performed under storage conditions (stressed conditions: 40° C./RH 75±5%) in accordance with the ICH guideline recommendations and drug stability test regulations.

From the results in Table 17 above comparing Examples 41 and 42, it can be confirmed that there is no difference in stability depending on the content of tegoprazan in the prepared tegoprazan enteric tablet.

In addition, as shown in Table 18 above, as a result of comparing the stability of each of tegoprazan and clopidogrel hydrogen sulfate, it can be confirmed that the stability of the tablet-in-tablet formulation was maintained by preventing physical contact between the two components through the enteric coating layer, etc. positioned between the core and the shell of the tablet-in-tablet formulation.

### Experimental Example 4-2. Evaluation of Dissolution Tests for Tablet-in-Tablet Formulations

For the Examples 43, 44, 46 and 47 prepared according to Table 16 above, dissolution tests were performed under the following conditions: 0.01N HCl and pH6.8 phosphate buffer; USP) apparatus 2; 50 rpm; 900 mL medium; HPLC analysis.

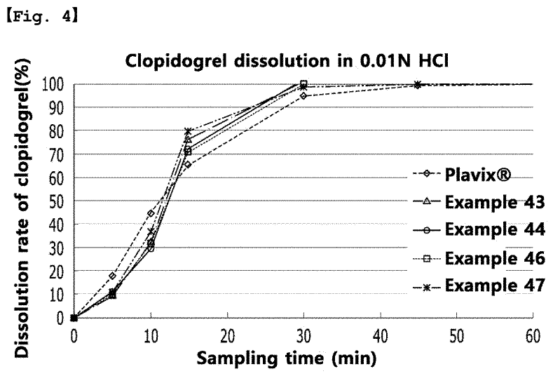

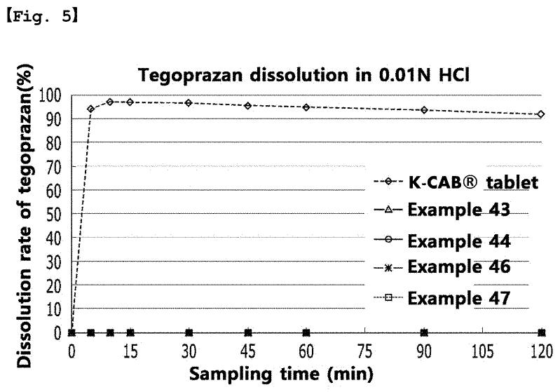

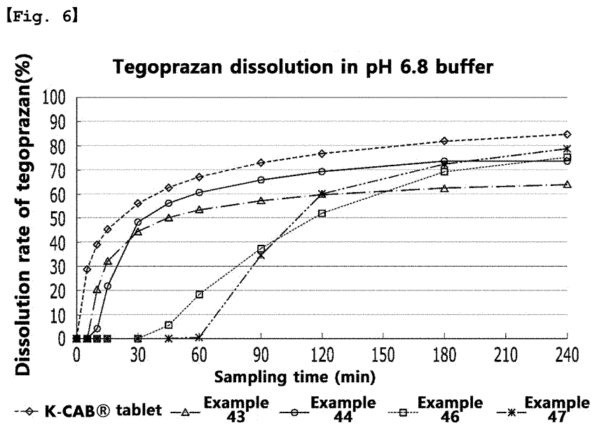

Specifically, the acid resistances of the tablet-in-tablet formulations of Examples 43, 44, 46 and 47 in 0.01N HCl were evaluated, and then the tablet-in-tablet formulations of Examples 43, 44, 46 and 47 subjected to the acid resistance evaluation were dosed to pH6.8 (alkaline condition) phosphate buffer and were additionally subjected to dissolution tests. The results are shown in FIG. 4 (a dissolution graph of clopidogrel in 0.01N HCl), FIG. 5 (a dissolution graph of tegoprazan in 0.01N HCl) and FIG. 6 (a dissolution graph of tegoprazan at pH 6.8).

From the results in FIGS. 4 to 6, it could be confirmed that the tablet-in-tablet formulations of Examples 43, 44, 46 and 47 of the present disclosure allowed only clopidogrel to be selectively dissolved in the gastric juice condition. In addition, it can be confirmed that, in the case of the tablet-in-tablet formulations of Examples 43, 44, 46 and 47, the tegoprazan enteric tablet which is the core tablet maintained its shape without breaking of the coating layer thereof after the acid resistance evaluation in 0.01N HCl, and tegoprazan was dissolved as a result of evaluating dissolution thereof in the pH6.8 (alkaline condition) phosphate buffer (FIG. 6).

Therefore, it can be seen that the tablet-in-tablet formulations of the present disclosure allow only clopidogrel to be selectively dissolved in the gastric juice condition, and delayed-release tegoprazan, suggesting that the excellent efficacy of clopidogrel of the present disclosure is maintained.

1. A pharmaceutical composition comprising:
a first compartment comprising a first active ingredient; and
a second compartment comprising a second active ingredient,
wherein the first active ingredient is delayed-released, and the second active ingredient is released immediately;
wherein the first active ingredient is tegoprazan, an optical isomer thereof, a pharmaceutically acceptable salt thereof, a hydrate or solvate thereof, or a mixture thereof; and
wherein the second active ingredient is clopidogrel, an optical isomer thereof, a pharmaceutically acceptable salt thereof, a hydrate or solvate thereof, or a mixture thereof.

2. The pharmaceutical composition of claim 1, wherein the first compartment comprising the first active ingredient and the second compartment comprising the second active ingredient are co-administered.

3. The pharmaceutical composition of claim 2, wherein the first compartment is a unit dosage form comprising the first active ingredient, and the second compartment is a unit dosage form comprising the second active ingredient.

4. The pharmaceutical composition of claim 3, wherein the unit dosage form comprising the first active ingredient comprises a particle comprising:
a core comprising the first active ingredient; and an enteric coating layer positioned on the core and surrounding the core.

5. The pharmaceutical composition of claim 4, wherein the particle is a tablet, a granule, a pellet, or a mixture thereof.

6. The pharmaceutical composition of claim 4, wherein the particle in the unit dosage form comprising the first active ingredient further comprises at least one additional coating layer.

7. The pharmaceutical composition of claim 3, wherein the unit dosage forms comprising the first active ingredient and the second active ingredient, respectively, are tablets or capsules which are administered independently of each other.

8. The pharmaceutical composition of claim 1, wherein the pharmaceutical composition is a complex in a unit dosage form comprising both the first active ingredient and the second active ingredient.

9. The pharmaceutical composition of claim 8, wherein the unit dosage form comprises: a first layer which is the first compartment comprising the first active ingredient; and a second layer which is the second compartment comprising the second active ingredient.

10. The pharmaceutical composition of claim 9, wherein the unit dosage form comprises a particle comprising:
a first layer, which is the first compartment comprising the first active ingredient, comprising: a core comprising the first active ingredient; and an enteric coating layer positioned on the core and surrounding the core; and
a second layer positioned on the enteric coating layer and surrounding the enteric coating layer and containing the second active ingredient.

11. The pharmaceutical composition of claim 10, wherein the particle is a tablet, a granule, a pellet, or a mixture thereof.

12. The pharmaceutical composition of claim 10, wherein the particle in the unit dosage form further comprises an isolation layer positioned between the first layer and the second layer and preventing contact between the first active ingredient and the second active ingredient.

13. The pharmaceutical composition of claim 8, wherein the unit dosage form comprises:
a first compartment which is a particle comprising the first active ingredient; and a second compartment which is a particle containing the second active ingredient,
wherein the first compartment which is a particle comprising the first active ingredient is a particle comprising:
a core comprising the first active ingredient; and
an enteric coating layer positioned on the core and surrounding the core.

14. The pharmaceutical composition of claim 13, wherein the particle comprising the first active ingredient further comprises an isolation layer positioned on the enteric coating layer.

15. The pharmaceutical composition of claim 13, wherein the particle comprising the first active ingredient and the particle comprising the second active ingredient are each independently a tablet, a pellet or a granule.

16. A complex comprising:
a first compartment comprising a first active ingredient;
a second compartment comprising a second active ingredient; and
an isolation layer preventing contact between the first active ingredient and the second active ingredient,
wherein the first active ingredient is tegoprazan, an optical isomer thereof, a pharmaceutically acceptable salt thereof, a hydrate or solvate thereof, or a mixture thereof, and
the second active ingredient is clopidogrel, an optical isomer thereof, a pharmaceutically acceptable salt thereof, a hydrate or solvate thereof, or a mixture thereof.

17. The complex of claim 16, wherein the isolation layer is positioned between the first compartment comprising the first active ingredient and the second compartment comprising the second active ingredient.

18. The complex of claim 16, wherein the complex comprises:
a first layer which is the first compartment comprising the first active ingredient;
the isolation layer formed on the first layer and preventing contact between the first active ingredient and the second active ingredient; and
a second layer which is the second compartment comprising the second active ingredient.

19. The complex of claim 18, wherein the complex is a tablet.

20. The complex of claim 19, wherein the tablet is a multilayered-tablet,
wherein the first layer in the multilayered-tablet comprises a granule comprising the first active ingredient,
wherein the second layer in the multilayered-tablet comprises a granule comprising the second active ingredient, and
wherein the isolation layer in the multilayered-tablet comprises a granule comprising a pharmaceutically acceptable additive.

21. The complex of claim 20, wherein the granule comprising the first active ingredient further comprises an enteric coating layer.

22. The complex of claim 16, wherein the complex is a multilayered-tablet, wherein the multilayered-tablet comprises:
a core which is the first compartment comprising the first active ingredient;
an isolation layer positioned on the core and comprising a pharmaceutically acceptable additive; and
a second compartment positioned on the isolation layer and comprising the second active ingredient.

23. The complex of claim 22, wherein the multilayered-tablet further comprises an enteric coating layer between the core and the isolation layer.

24. The complex of claim 16, wherein the complex is a capsule.

25. The complex of claim 24, wherein the capsule comprises a particle comprising:
a core which is the first compartment comprising the first active ingredient;
an isolation layer which is positioned on the core and a coating layer comprising a pharmaceutically acceptable additive; and
a second compartment positioned on the isolation layer and comprising the second active ingredient.

26. The complex of claim 25, wherein the particle is a tablet, a granule or a pellet, and the capsule is filled with the tablet, granule, pellet, or mixture thereof.

27. The complex of claim 25, wherein the particle further comprises an enteric coating layer for delayed release of the first active ingredient.

28. The complex of claim 16, wherein the complex is a capsule,
wherein the capsule comprises: a first compartment which is a particle comprising the first active ingredient; and a second compartment which is a particle comprising the second active ingredient,
wherein the first compartment which is a particle comprising the first active ingredient is a particle comprising:
a core comprising the first active ingredient; and
an isolation layer positioned on the core and surrounding the core.

29. The complex of claim 28, wherein the first compartment which is a particle comprising the first active ingredient further comprises an enteric coating layer.

30. The complex of claim 29, wherein the enteric coating layer is positioned between the core and the isolation layer.

31. The complex of claim 28, wherein the particle comprising the first active ingredient and the particle comprising the second active ingredient are each independently a tablet, a pellet or a granule.

32. A pharmaceutical composition for preventing or treating gastrointestinal disorders caused by administration of clopidogrel, an optical isomer thereof, a pharmaceutically acceptable salt thereof, a hydrate or solvate thereof, or a mixture thereof, comprising tegoprazan, an optical isomer thereof, a pharmaceutically acceptable salt thereof, a hydrate or solvate thereof, or a mixture thereof.

33. The pharmaceutical composition of claim 32, wherein the tegoprazan, optical isomer thereof, pharmaceutically acceptable salt thereof, hydrate or solvate thereof, or mixture thereof is delayed-released.

34. A pharmaceutical composition for inhibiting gastric acid secretion, comprising tegoprazan, an optical isomer thereof, a pharmaceutically acceptable salt thereof, a hydrate or solvate thereof, or a mixture thereof,
wherein the pharmaceutical composition is co-administered with clopidogrel, an optical isomer thereof, a pharmaceutically acceptable salt thereof, a hydrate or solvate thereof, or a mixture thereof.

35. The pharmaceutical composition of claim 34, wherein the tegoprazan, optical isomer thereof, pharmaceutically acceptable salt thereof, hydrate or solvate thereof, or mixture thereof is delayed-released.

36. A pharmaceutical combination comprising:
tegoprazan, an optical isomer thereof, a pharmaceutically acceptable salt thereof, a hydrate or solvate thereof, or a mixture thereof; and
clopidogrel, an optical isomer thereof, a pharmaceutically acceptable salt thereof, a hydrate or solvate thereof, or a mixture thereof.

37. A method for preventing or treating gastrointestinal disorders caused by administration of clopidogrel, an optical isomer thereof, a pharmaceutically acceptable salt thereof, a hydrate or solvate thereof, or a mixture thereof, comprising administering to a subject a pharmaceutically effective amount of the pharmaceutical composition according to any one of claims 1 to 15 or the complex according to any one of claims 16 to 31.

38. A method for preventing or treating thrombosis-related diseases, comprising administering to a subject a pharmaceutically effective amount of the pharmaceutical composition according to any one of claims 1 to 15 or the complex according to any one of claims 16 to 31.

39. Use of the pharmaceutical composition according to any one of claims 1 to 15 or the complex according to any one of claims 16 to 31 for preventing or treating gastrointestinal disorders caused by administration of clopidogrel, an optical isomer thereof, a pharmaceutically acceptable salt thereof, a hydrate or solvate thereof, or a mixture thereof.

40. Use of the pharmaceutical composition according to any one of claims 1 to 15 or the complex according to any one of claims 16 to 31 in the manufacture of a medicament for preventing or treating gastrointestinal disorders caused by administration of clopidogrel, an optical isomer thereof, a pharmaceutically acceptable salt thereof, a hydrate or solvate thereof, or a mixture thereof.

41. Use of the pharmaceutical composition according to any one of claims 1 to 15 or the complex according to any one of claims 16 to 31 for preventing or treating thrombosis-related diseases.

42. Use of the pharmaceutical composition according to any one of claims 1 to 15 or the complex according to any one of claims 16 to 31 in the manufacture of a medicament for preventing or treating thrombosis-related diseases.
